(19) United States
(12) Patent Application Publication
(10) Pub. No.: US 2026/0033737 A1
(43) Pub. Date: Feb. 5, 2026

(54) WEARABLE MONITOR FOR THE EARLY DETECTION OF INTERNAL BLEEDING EVENTS AFTER CARDIAC CATHETERIZATION

(71) Applicant: HemaSense, Inc, Chicago, IL (US)

(72) Inventors: Benjamin Michael Trapp, Scottsdale, AZ (US); Cody L. Hartman, Flagstaff, AZ (US); Nathan Friedman, Flagstaff, AZ (US); Anita W. Asgar, Chicago, IL (US)

(21) Appl. No.: 19/353,143

(22) Filed: Oct. 8, 2025

Related U.S. Application Data
(63) Continuation of application No. PCT/US25/22085, filed on Mar. 28, 2025.
(60) Provisional application No. 63/571,078, filed on Mar. 28, 2024.

(51) Int. Cl. A61B 5/0536; A61B 5/00

(52) U.S. Cl. CPC A61B 5/0536; A61B 5/4836; A61B 5/6833; A61B 5/7271; A61B 5/746; A61B 2560/0223; A61B 2560/0468; A61B 2562/046; A61B 2562/066

(57) ABSTRACT

A wearable monitor is configured for early detection of internal bleeding events and particularly hematomas after vascular catheterization including cardiac catheterization. The wearable monitor is placed over an access location following the vascular catheterization procedure and the wearable monitor system periodically runs diagnostic test to detect changes in the tissue around the vascular access location. An emitter device may input an emitter input into the tissue and a detector device may then measure the emitter input after it has passed through the tissue, an emitter output. A computer may receive the data from the detector device and operate a program that determines changes in the tissue and in particular, pooling of blood in the tissue. The emitters may be electrodes that input an electromagnetic input, or an ultrasound emitter that inputs ultrasound sound waves, or a Near IR emitter that emits Near IR electromagnetic radiation.

### CROSS REFERENCE TO RELATED APPLICATIONS

This application is a continuation of international patent application No. PCT/US2025/022085, filed on Mar. 28, 2025 and currently pending, which claims the benefit of priority to U.S. provisional patent application No. 63/571,078, filed on Mar. 28, 2024; the entirety of all priority applications are hereby incorporated by reference herein.

### BACKGROUND OF THE INVENTION

### Field of the Invention

The invention relates to a wearable monitor for the early detection of internal bleeding events and particularly hematomas after vascular catheterization including cardiac catheterization. The wearable monitor may be placed over an access location following the vascular catheterization procedure and the wearable monitor system may periodically run a diagnostic test to detect changes in the tissue around the vascular access location. The wearable monitor may alert the patient or caretaker to changes to tissue that correspond to a bleeding event.

### Background

Cardiac catheterization requires that an introducer or sheath penetrate the skin and puncture a vessel (vein or artery). A catheter is then inserted through the sheath for a variety of procedures. Upon removal of the catheter and sheath, there is a risk of bleeding from the tissue and/or the vessel that can cause serious complications. A hematoma may form and may go undetected for a period of time as the pooling of blood under the skin may not initially cause any pain, swelling or infection. These hematomas may not be detected until they are clinically manifested in symptoms such as pain, swelling, or loss of blood pressure, etc.

Early detection of vascular access site bleeding or hematoma formation would facilitate more rapid treatment and the prevention of serious bleeding complications even prior to significant clinical manifestations. Patients are at an increased risk for bleeding complications during the 24 hour period following intervention and prior to hospital discharge.

### SUMMARY OF THE INVENTION

The invention is directed to a wearable monitor for the early detection of internal bleeding events and particularly hematomas after vascular catheterization including cardiac catheterization. The wearable monitor may be placed over an access location following the vascular catheterization procedure and the wearable monitor system may periodically run a diagnostic test to detect changes in the tissue around the vascular access location. An emitter device may generate an emitter input into the tissue and a detector device may then measure the emitter signal as a detection signal after it has passed through the tissue, an emitter output. An emitter input may be a pulse of energy or an oscillating input having a variation in magnitude or amplitude and/or frequency. A particular power level including amplitude and frequency may be more effective at detecting a bleeding event than other power level emitter input and the controller or computer of the wearable sensor system may determine what type of signal is best for detection of tissue parameters and changes in the tissue, such as pooling of blood therein. A controller may include a computer to receive the data from the detector device and operate a program that determines changes in the tissue and in particular, pooling of blood in the tissue. The controller may initiate an alert device in the event that a bleeding event has occurred. Early detection of a bleeding event may aid in preventing serious complications. The wearable monitor system may include an attachment component such as an adhesive and/or straps and/or bands to allow the patient to wear the wearable monitor even during ambulatory events, such as when going to the bathroom.

An exemplary wearable sensor system, or monitor system, includes a wearable monitor and an attachment component to enable detachable attachment of the wearable monitor on tissue, such as near the incision of a vascular catheterization. The wearable monitor may extend from an incision location along an access track and over a vessel puncture location. An access track is the track in the tissue from the skin incision to the vessel puncture location made by the introducer or access sheath. This area may be most suspectable to a bleeding event and development of a hematoma following vascular catheterization.

A plurality of emitter devices and/or detector devices may be configured over this wearable monitor to produce a monitored area for detecting a bleeding event and/or hematoma. In some cases, the emitter devices may act also as a detector device and therefore are referred to herein as transducers, as they may input or detect signals. The monitored area, and/or area of the wearable monitor may be about 25 cm² or more, about 50 cm² or more, about 75 cm² or more, about 100 cm² or more, about 150 cm² or more, about 200 cm² or more and any range between and including the areas provided. A larger area may provide details over a larger area of tissue, however, some emitter inputs may lose fidelity in detection of a bleeding event or hematoma when measured over too large of an area. It is preferred to have the monitored area extend around the access area, or from one side of the skin incision to beyond the vessel puncture. The wearable monitor may be rectangular in shape and have a side dimension of about 5 cm or more, about 10 cm or more, about 15 cm or more, about 20 cm or more. The wearable monitor may have a length along a length axis of about 5 cm or more, about 10 cm or more, about 15 cm or more, about 20 cm or more and a width, orthogonal to the length that may also be about 5 cm or more, about 10 cm or more, about 15 cm or more, about 20 cm or more.

The emitter devices and/or detector devices may be configured over the area or the wearable monitor or may extend along a side dimension as provided. The wearable device may also provide a specific shape to better conform to the anatomy, for example, a wearable monitor for a femoral access site may have a relatively narrow bottom area that extends on the leg and crosses the crease between the leg and abdomen before it widens on the abdomen.

The wearable monitor may also provide a geometry to allow a bandage to be applied and/or changed over the skin incision without having to reposition or remove the wearable monitor or patch. The wearable monitor may have an open area that may be a closed open area such as a window within the wearable monitor, which may be oval or round in shape or rectangular in shape and may extend along a length axis of the wearable monitor, wherein the length axis may be centrally located on the wearable monitor and configured to extend over an access track. The wearable monitor may have an open area that is contiguous with the perimeter of the wearable monitor wherein the open area is V-shaped or U-shaped, with the narrow portion of the V-shaped wearable monitor configured near the tissue incision and the wider portion near the vessel puncture. Also, a wearable monitor may have an irregular shape and may have monitoring hubs configured to form an open area over the access location. Also, a wearable monitor may be configured such that lead lines to the transducers, emitter and detectors, do not cross over the access location or access track. An open area in the wearable monitor may also allow the skin incision to be visualized and/or to allow for treatment in the event of a bleeding event (eg. applied pressure). An open area may be large enough to effectively monitor for any complications in the vessel access area including the skin incision, access track and vessel puncture and therefore may have dimensions of about 1 cm wide by about 3 cm long and may be from about 1 cm to about 2 cm wide and 3 cm to 10 cm long, or encompass an area of about 3 cm² to about 30 cm². Any dimension of the open area may be at least 1 cm and preferably 2 cm or more. An open area that is too small would not allow effective examination of the access site. A window type open area is enclosed by the monitor patch and a slot type open area is formed by the shape of the wearable patch that is open to the open area along a portion of the open area.

A wearable monitor has a skin interface side configured to be placed against a person's skin and an exposed side, opposite the skin interface side. The wearable monitor may include a monitor patch, a component to which the emitter devices and/or detector devices are attached. The monitor patch is a means for holding a series of emitter devices and detector devices in a position that allows an area of interest, such as a vessel access site, to be interrogated via an emitter input and detection of this emitter input by a detector device. The wearable monitor may monitor for changes in a detection signal as it interrogates the tissue in the area of interest. A monitor patch may be planar and include a fabric or a planar piece of material, such as a urethane, polyester or silicone film and may also include a non-woven, knitted or woven fabric made of polyester, nylon or polyolefin. The layers may also include an adhesive such as an acrylic or silicone layer which promotes fixation to the skin. The monitor patch may be a material that does not interfere with the emitter input or detection signal to ensure high resolution of detection. A monitor patch may be electrically non-conductive having a resistivity of about 10⁸ ohm-m or more.

The emitter devices and/or detector devices may be configured in a row wherein a plurality of emitter devices and/or detector devices are aligned along the wearable monitor positioned to interrogate the area of interest. In an exemplary embodiment the area of interest comprises the access track, including the skin incision and vessel puncture. In an exemplary embodiment, the emitter input passes through tissue spanning the access track and a detector device detects changes in a detection signal from the emitter input passing across this access track region. In an exemplary embodiment, emitter devices are configured to introduce emitter input on a first side of the access track and detector devices are configured to detect a detection signal from the emitter input that crosses to a second side of the access track. In an exemplary embodiment, emitter devices are configured to introduce an emitter signal on one side or on opposing sides of the access track and may extend substantially over the length of the access track. The emitter devices may extend on the skin over or along the access track, wherein a first emitter device is configured close to the incision in the skin, and a second emitter device is configured over the vessel puncture or beyond the vessel puncture from the skin incision. The emitters may span over the entire access track from the vessel access site or incision in the skin to a location on the skin over the vessel puncture past the vessel puncture with respect to the skin incision. A plurality of emitter devices may be configured to input signals that cross over the access track such that at least 75% of the length of the access track may be interrogated by the plurality of emitter and detector devices. In some embodiments, emitter and detector devices may extend past the vessel puncture site to monitor for bleeding events further offset or past the vessel puncture site with respect to the skin incision, such as a retro-peritoneal hematoma.

An interrogation axis, is an axis along with an emitter input travel through the tissue for detection and therefore an interrogation axis may extend from an emitter to a detector or in the case of a four-point probe, may extend between a first and second emitter, wherein the detector devices of the four point probe are configure to measure the emitter input between the two emitter devices and may be aligned with the interrogation axis or offset therefrom.

When the emitter devices are electrodes and the detector device are electrodes, a four-point probe configuration may be employed to reduce contact resistance between the electrodes and the skin tissue for monitoring changes in impedance in the tissue, such as for Electrical Impedance Tomography (EIT). In a four-point probe configuration, a pair of electrode emitters are configured to input an electrical signal into the tissue with one electrode emitter inputting the electrical signal and one electrode emitter acting as a return or conductor for this input electrical signal, and a pair of electrode detectors are configured to detect this emitter input electrical signal, by measuring a drop or change in the electrical signal from a first and second electrode detector. In a four-point probe, the electrode detectors are configured between the two electrode emitters and the interrogation axis is between the two electrode emitters. The four electrodes may be configured in a line along the interrogation axis, or the electrode emitters may be offset from the interrogation axis.

An electrical signal may have a frequency of about 200 kHz or less, about 150 kHz or less, about 100 kHz or less and any range between and including the values provided. The electrical signal may be applied at a voltage to effectively drive the electrical signal into the tissue and through the tissue from a first or input electrode emitter to a second or return electrode emitter.

In an exemplary embodiment, the area of interest may be interrogated using a series of emitter devices and detector devices that directionally orient and control the spacing of the emitter signal over the area of interest. Changes in emitter signals that are directionally oriented and spaced may allow for interpretation of spatial and temporal changes that correspond to the direction and rate of accumulating blood or tissue changes. In an exemplary embodiment, there is at least 2 rows of emitter and detector devices that are oriented parallel to each other allowing for a rate of growth to be derived.

For application of a wearable monitor over a vessel access site, an array of emitters and detectors may enable interrogation along an interrogation axis that is across the access track and/or along one or both sides or the access track. It may be preferred to have a plurality of interrogation axes that cross the access track, such as at least one closer to the skin incision location and another closer to the vessel puncture location or beyond the vessel puncture from the skin incision. This combination of interrogation axes may provide for more reliable and quicker determination of a bleeding event.

An exemplary wearable monitor may include a grid of emitter devices and/or detector devices that extend over a monitored area of the wearable monitor. The grid may include evenly spaced apart emitter devices and/or detector devices such as in a series of rows and columns of emitter devices and/or detector devices. The emitter devices and detector devices may be configured in evenly spaced rows and/or columns on the wearable monitor.

An exemplary wearable monitor system may be configured to activate one or more of a plurality of emitter devices and/or detector devices to conduct a measurement for more accurate detection of a bleeding event and hematoma. For example, one or more emitter devices may be activated to initiate a respective emitter input into the tissue and one or more detector devices may be activated for measurement or detection of the emitter signal. The emitter devices may be on one side or opposing sides of the access track and likewise, the detector devices may be on one side or opposing sides of the access track. The emitter and/or detector devices that are activated may be along the access track, from the vessel access site or incision in the skin to the vessel puncture and the distance from the access track may be changed to obtain data or a measurement deeper in the tissue. The emitter and/or detector devices may be selectively enabled or disabled as a bleed grows to obtain information about the rate and direction of the bleed. The emitter and/or detector devices that are activated may form a V-shape along the access track with emitter and/or detector devices having a greater offset distance adjacent to the vessel puncture than offset distances of devices more adjacent to the skin incision.

Emitter devices and detector devices may be activated or utilized to detect changes in the tissue at various depths within the tissue. A first set of emitter devices and/or detector devices may be activated with a first offset distance between them and then a second set may be activated with a second offset distance that is greater than the first offset distance by 10% or more, 20% or more, 35% or more, 50% or more, or even 75% or 100% or more. A greater distance offset may provide different data about the underlying tissue. A greater offset distance may be used to interrogate tissues at a deeper depth, to monitor for bleeding occurring deeper in the tissue. A lesser offset distance may be used to interrogate tissue at a shallower depth, for instance to avoid interrogating higher conductivity anatomies like underlying muscle that may lessen the sensitivity of the desired measurements.

An exemplary wearable monitor system may use less power by activating only a portion of the plurality of emitter devices and/or detector devices, such as one or more, two or more, three or more, five or more, ten or more and any range between and including the number of activated emitters and/or detector devices. Put another way, a percentage of the total emitter devices and/or detector devices of the plurality of emitter and detector devices respectively may be activated for effective detection of a bleeding event and this percentage may be about 50% or less, 30% or less, about 20% or less, about 10% or less, or even 5% or less. For example, a monitoring array may have 20 emitters and only five of the emitters, or 25% of the total number of emitter devices may be activated to effectively detect a bleeding event. The number of emitters and detector devices required and their location may be determined after an initial calibration to determine an access track and/or vessel puncture location. Power consumption may be greatly reduced by this selective activation of a portion of the emitter devices and/or detector devices.

A wearable monitor may include a plurality of emitter devices and/or detector devices, or transducers, to provide effective monitoring of the tissue, such as about two or more, about four or more, about six or more, about ten or more, about 15 or more, about 20 or more, about 40 or more and any range between and including the values provided. A higher number of emitter devices and/or detector devices may enable more combinations of interrogation paths or interrogation axes to provide better detection of a bleeding event and formation of a hematoma. A controller may activate one or more emitters and one or more detector devices to produce the most effective detection along an access track. The specific emitter devices and detector devices activated for this purpose may be different from patient to patient depending on the length and depth of the access track and positioning of the wearable monitor over the venous access area. Having a higher number of emitters may enable more effective and earlier detection of a bleeding event.

The higher number of emitter and/or detector devices allows for more precise measurement by increasing the resolution of the signals by interrogating a smaller portion of the area by each detector and emitter combination. A higher number of emitter and/or detector devices may also allow for more precise compensation for any anticipated or unanticipated signal interference or degradation, including patient movement, change in moisture on the skin surface, or movement of the wearable monitor due to other external factors.

An exemplary wearable monitor system includes a computer that operates a computer program, which is non-transitory or a non-transitory medium, to analyze the data from the detector devices. The computer may be configured on the wearable monitor or may receive a wireless signal from the wearable monitor with the data from the detector devices. A computer may utilize a microprocessor to run computer programs. A wireless signal transmitter or transceiver may be configured on the wearable monitor to communicate with the computer and/or an alert device. In some cases, the computer may provide instructions to the wearable monitor or controller of the wearable monitor for the activation of emitter devices and/or detector devices. Also, the computer may increase the frequency of measurements when an initial anomaly or change in the tissue is detected. The frequency may increase by being more than doubled, or more than tripled, for example, when a change in the tissue is detected. The frequency may be greater when there is a higher risk and/or likelihood of a bleeding anomaly and then decreased as the risk and/or likelihood decreases. The measurement frequency may be higher for the first hour following a procedure than after ten hours following the procedure, higher for, the first two hours or less, the first four hours or less, etc. and decreased after these initial time frames. The measurement frequency may be about one minute or less, about five minutes or less, about 15 minutes or less, about 30 minutes or less about one hour or less and any range between and including the time intervals for the frequency provided. A more frequent rate of measurement may provide quicker detection of changes in the tissue but may also more quickly drain a battery.

A threshold value for a detection signal may be an absolute value but preferably it is a threshold value based on an initial value when the wearable monitor is implemented such as over a vessel access site. The wearable monitor may be placed over tissue before access to the vessel to determine a baseline or a baseline may be established when the wearable monitor is placed on the tissue after a vessel access procedure. A change in a baseline value of about 5% or more, about 10% or more, or even 25% or more may indicate a threshold value. For example, a drop in impedance of 10% from a baseline impedance value may initiate an alert or alarm as this may indicate a bleeding event. Also, a rate of change of a detection signal may be a factor that the controller used to determine if an alert or alarm is activated.

### Alerts and Alarms

A wearable monitor system may include an alert device, a device that will alert staff and/or the patient that there may be a bleeding event or a hematoma formed. An alert device may be a light device that emits a light or a sound device that emits a sound or alarm. The alert device may be on the wearable monitor or may be configured where staff will be alerted. An alert device may be configured on a wireless mobile device, such as a mobile phone. An alert device may be configured as a part of an existing monitoring system that is used for other patient monitoring purposes, for example an oxygen monitor, a heart rate monitor, a blood pressure monitor, or other patient status monitoring systems. An alert device may be configured as a part of an existing patient data and information system, such as an electronic health record system. The system may already have alert capabilities in place which the monitor system may communicate with in order to provide alerts through existing systems. An alert may be activated on an existing device already used in the patient monitoring workflow, for example a computer, speaker, screen, or other device at a nurse's station, or on a computing tablet or on a mobile phone which is transported with an individual.

An exemplary alert device may produce a different alert depending on the severity of the detected bleeding. If the rate of growth of pooled blood is above a first threshold rate indicating a minor bleeding event, a first type of alert signal, light or sound, may be produced by the alert device but if the rate of growth of the pooled blood is above a second threshold indicating an emergency, a second type of alert signal, light and/or sound, may be produced by the alert device. Different sounds or visual cues could indicate the severity of the bleeding complication (eg. the size or rate of growth of a hematoma) based on an output from an algorithm. A sound that increases in pitch or intensity with a more severe bleed may be used. Or a light that changes from green to yellow to red indicating a scale of severity may be used. Other more sophisticated methods could be employed where a message is delivered over a network or through wireless communication to an area where nurses view patient status or to a pager or phone on a nurse.

An alert device may be configured to produce an alert signal, light and/or sound if the wearable monitor is detected as being dislodged from the patient. The wearable monitor may run a monitoring scan by activating an emitter and detector and the signal returned, or no input being detected, may indicate that the wearable monitor has become dislodged and is not properly attached to the patient over the access track. The alert signal for this type of event may be different than an alert signal for detection of a bleeding event.

A wearable monitor may be portable and not require any power of connection cables for operation. A wearable monitor may include a battery to power a controller, the computer, the wireless signal transceiver, alert device and the emitter devices and/or detector devices. It is anticipated that the device will need to maintain functionality for at least 24 hours after application and so the power source must be sized appropriately to enable that timeframe.

Prior to a vascular catheterization procedure, the wearable monitor may be placed over the access location and a baseline value for the tissue may be measured and saved by the computer. After a vascular catheterization procedure, the wearable monitor may be placed over the access location and span from the skin incision to over the vessel puncture, along the access track. A post procedure baseline of the tissue may be measured by activation of the emitter devices and detector devices. These baselines may be used by a computer program to discern a change in the tissue that may be attributed to a bleeding event and/or a hematoma.

The wearable monitor may include an open area through which direct access to the skin incision may be provided. This open area may provide a means for aligning the wearable monitor to the access track. The open area, or window may also provide access to apply a bandage or gauze to the incision in a manner that does not disrupt or require movement of the wearable monitor. The open area may also provide an area which allows for visualization and/or treatment of the vessel track without having to disrupt or move the wearable monitor.

### Detection Requirements and Dimensions

An exemplary wearable monitor system may effectively detect blood leaving the vessel, pooling blood, and/or the growth in size of pooled blood or hematoma size. For instance, a 1 cm³ amount of pooled blood may not be clinically relevant if it is stable, but if it is growing, and growing at a rate above a predetermined threshold, it should be immediately flagged for evaluation, and medical staff should be automatically alerted. The computer of the wearable monitor system may evaluate data from the detector devices and determine the size of pooling blood and may compare results from successive measurements to determine a rate of growth of the pooling blood. To detect growth of a hematoma, both temporal and spatial resolution at a useful depth is required. Temporal and spatial resolution may be used to detect size growth of the hematoma, and/or may be utilized to detect a direction and rate of progression of the blood accumulation. Direction and rate of progression may be used to predict the likelihood of blood traveling into the retroperitoneal space, down the leg towards the foot, or in other directions. Bleeding complications may occur at the vessel puncture or along the vessel in proximity to the sheath entry site. Bleeding could occur in the depth of the tissue, such as along the skin incision at a depth of 0 cm or at depths from the skin surface of about 2 cm or more, about 5 cm or more, about 7.5 cm or more or even 10 cm or more in larger patients. Retroperitoneal bleeding may also occur into the retro-peritoneal space which can be about 15 cm from the incision site and posterior, rendering it undetectable with conventional ultrasound imaging capability. The exemplary wearable monitor system may be configured to detect a bleeding event, or hematoma at a depth from the skin surface of about 1 cm or more, about 2 cm or more, about 5 cm or more, about 7.5 cm or more, about 10 cm or more, about 12.5 cm or more, about 15 cm or more and any range between and including the depth of monitoring values provided. The wearable monitor may be configured to detect the direction of progression of the bleeding event. The arrays may be arranged in a grid pattern, a series of lines, or other patterns which will allow detection of a direction of hematoma growth and rate of progression to be derived over time.

The wearable monitor system combines the ability to be positioned on a patient (wearable) and provides temporal and spatial resolution at a useful depth. The emitter devices may be Ultrasound, Near-Infrared (NIR), and Electrical Impedance Tomography (EIT). An ultrasound emitter device emits sound waves into the tissue as the emitter input and an ultrasound detector device detects a detection signal of sound waves that has passed through the tissue from the one or more ultrasound emitter devices. A Near-Infrared (NIR) emitter device emits NIR light into the tissue as the emitter input and a NIR detector device detects a detection signal of NIR light that has passed through the tissue from the one or more NIR emitter devices. An EIT emitter device, or an electrode emitter emits electromagnetic energy (current and voltage), an electrical signal as used herein, into the skin as the emitter input and a EIT detector device detects a detection signal of electromagnetic energy, electrical signal, from the EIT emitter device.

A wearable monitor system may include other modalities for measurement or detection of blood pooling, such as measuring the surrounding tissue for changes. For instance, a strain gauge could be used to measure a hematoma that manifests itself as a bulge or swelling of the skin. A pressure gauge measuring intracellular pressure or tissue pressure could also indicate swelling or a growing hematoma. These devices may be combined with a wearable monitor system.

The wearable monitor control algorithm may also be configured to take in other input to help determine if changes in impedance are due to pooling of blood or other artifacts. As an example, data from an accelerometer could be used to predict changes in impedance that are due to movement that would otherwise be interpreted as a potential bleeding event. Data input of systemic blood pressure may be used to triage the severity of a bleed where a drop of impedance with a corresponding drop in systemic blood pressure could trigger a very high severity alarm with a high risk to the patient.

### Configuration of Sensor Arrays and Calibrating

Ultrasound, NIR and EIT all involve the positioning of emitter devices and detector devices around or near the measurement location for proper detection. These emitters and detectors must be placed specifically to allow for monitoring at the correct depth with the correct focus or spatial and temporal resolution. For instance, the angle of the emitter or detector may need to be tuned or the distance between emitter and detector may need to be tuned to focus on the site at risk for the bleeding complication.

The ability to tune and position the emitters and detectors correctly is problematic. To aid in effective positioning, a wearable monitor may be applied while the introducer or access sheath is still in place and wearable monitor system may tune the sensors to the spatial positioning of the sheath (because the sheath is in the access track at high risk for bleeding and may be easily located with the monitoring system). A variety of emitter devices and detector devices may be activated until an effectively high resolution rendering of the location of the access track is detected. The introducer or sheath may have features to help in this alignment, such as materials reflective to ultrasound, of known impedance, or that absorb/reflect light at a certain wavelength. The emitter or detector devices can be tuned in angle, spacing, or by other means to narrow their field of view to the specific location corresponding to the sheath and access track and thus to areas at high risk for bleeding.

### Electrical Impedance Tomography (EIT)

Electrical Impedance Tomography (EIT) involves the interrogation of the tissue with current to measure the resulting voltage and losses induced by the current fields. EIT emitter devices and EIT detector devices may be electrodes that can be activated as one or the other as determined by the controller. An electrode may be a transducer and may act as an EIT emitter device and subsequently act as an EIT detector device in a subsequent measurement. In a four-wire impedance test or four-point probe impedance test, there are two emitter devices and two detector devices active during a single measurement. The current travels from one of the emitter devices to the other emitter device in a prescribed direction but that direction can be varied with a change in polarity. The two detector devices are separated from the emitter devices and are configured to measure the resulting voltage or electrical signal created from the applied current into the tissue. This measured voltage can be used to calculate impedance with a known applied current. The current path being interrogated from in plane impedance measurement using a substantially planar array of electrodes is dependent on the spacing of the electrodes. EIT emitter devices such as electrodes that are spaced further apart will interrogate deeper into the tissue. Tuning the electrode spacing to match the depth of tissue at high risk for bleeding complications is important to increase the probability of detection.

An exemplary wearable monitor may have a plurality of EIT emitter and detector devices that can be activated as required for effective measurement which eliminates the need to measure and then move the electrodes for subsequent measurements. The electrodes used for interrogation can be varied, allowing the system to be tuned to different depths. Once a desired depth is tuned (for example by looking for a strong signal from the sheath before it is removed), the electrode interrogation can cycle between a close spacing (small offset distance between activated electrodes) to a wider defined spacing to scan the depth of interest, including the access track through the tissue from the skin incision to the vessel puncture. When a change in impedance is detected with respect to the baseline tissue, signaling a potential for pooled blood, the signal can be closely monitored at neighboring depths to understand when the pool of blood is growing in size and at what rate. Electrodes with a smaller offset distance may be more effective to monitor for bleeding at an area close to the skin incision because the depth of measurement, or input signal depth, is smaller compared to electrodes that have a much higher offset distance.

As described herein, the electrodes may be configured in a ‘V’ shape from a ‘vertex’ near to or on a first side of the skin incision to a widening portion that extends from the vertex and this widening portion may extend or past a vessel punction. A length axis of the wearable monitor may extend centrally along the V-shape arrangement of electrodes and this V-shape may form an open area in the wearable monitor. This arrangement provides an increased depth of measurement along the access track. Also, the electrodes may have variations in the emitter output, such as the current applied to the skin along this V-shaped electrode array. To monitor for retroperitoneal hematomas, a separate array of sensors can be placed on the patient's abdomen or back where the depth may need to be increased further, resulting in an increased spacing of the electrodes at points furthest away from the vertex of a V-shaped activated electrode arrangement. There may also be instances where additional electrodes may be placed in a remote location from the proximity of the incision but may still be in communication with the wearable monitor or computer. For instance, a set of electrodes may be placed on the patient's back to further test for retroperitoneal bleeding while the electrodes on the front of the patient may evaluate for bleeding along the access track. Both sets of electrodes may be controlled by the controller of the wearable monitor system.

Sources of variability that may shift impedance values that are not related to bleeding need to be understood in a way where false alarms are not generated. For instance, changes in skin hydration, muscle flexion during walking, are known to shift impedance values but should not generate false alarms. A set of electrodes at a location separated from the wound and producing an input signal depth optimized for understanding skin hydration could be used as a ‘baseline’ or reference point that could be used to detect a new or emerging bleeding event.

### Near-Infrared (NIR) Spectroscopy

Near-infrared sensing relies on the penetration of near-infrared light wavelengths, a form of electromagnetic radiation, through various structures of the body, with a goal of reflecting certain wavelengths when a reflective substance is reached within the body. NIR emitters are typically light emitting diodes (LEDs) with a broad range of wavelengths, and then detectors (or photodiodes) which are tuned to a very narrow wavelength are used to measure the reflectance of the wavelength of interest. A single narrow wavelength detector could be used to selectively detect the presence of blood. For example, oxygenated blood has a high reflectance in the 800-900 nm range, so a detector in that range would have a high likelihood of detecting the presence of arterial blood. Unoxygenated blood has higher reflectance in the 700-800 nm range, and so a detector in that range would have a higher likelihood of detecting venous blood. Two different wavelength detectors could be used to interrogate both venous and arterial access points in the same device. Alternatively, only using a set of detectors that correspond to whether the procedure was a venous access or arterial access procedure may help down select what is being monitored and result in a more reliable result.

An array of NIR emitter devices such as LEDs may be used to bathe the area of interest in NIR light, with several detector devices interspersed within the array of emitter devices to create a flat sheet form factor. The distance between the detector and the LED can be increased to increase the depth of penetration of the emitter input or the input signal depth. The power output of the LED can also be used to increase depth of emitter input penetration.

It may be useful to have an array of both LEDs and detector devices that can be cycled through to interrogate various depths of tissue penetration as well as various locations along the access track. One of the known downsides of NIR spectroscopy measurements is the scattering of light as it penetrates deeper into the tissue, which can limit the spatial accuracy of the measurements. To overcome this limitation, the activation of the LEDS can be cycled through in a systematic pattern, and the measurements of intensity combined with the known locations of the detectors can be used to overlay datasets and create a more accurate map of the location of any detected blood or hematoma. A similar cycling of the LEDS and interrogation of the detectors could be used to calibrate or zero the device when it is first applied. Initial calibration may be done by using the NIR array to locate and detect the size of the native blood vessel and then use this reading as a baseline at the time point directly following sheath removal.

The use of pivoting or angled LEDs and/or detectors may help focus the monitoring in the site of interest. For instance, having a V-shaped rod of detectors, fixed to a patch in a manner that can be pivoted to focus the detector at the highest likelihood of bleeding complications may result in a more sensitive signal.

### Ultrasound Interrogation

Ultrasound imaging, also known as sonography, is a technique that uses sound waves to detect structures inside of the body and is the most common technology used to detect hematomas. An ultrasound emitter device is a transducer probe, which is placed directly on the skin. This probe can both send and receive high-frequency sound waves into and out of the body. As the sound waves travel inside the body, they hit different structures and tissues. Depending on the density and composition of these tissues, some of the sound waves are reflected back to the probe, while others continue to travel deeper. This reflection is called an echo and is typically reconstructed into a 2D image with varying greyscale colors. The transducer probe is also equipped to receive these echoes. Each echo arrives back at the probe at a different time and with varying strength, depending on how far away it reflected from and the characteristics of the tissue it bounced off.

The ultrasound signal can be used to detect pooled blood and calculate the distance to the border of the pooled blood based on the time it took for the echo to return and the strength of the signal. The return of subsequent echoes can then be used to determine whether the boundary of the pooled blood is progressing in size towards the skin (or away from the skin), putting the patient at a higher risk. The size and rate of growth of pooled blood or a hematoma may be monitored and measured using the ultrasound signal. In typical, manual Ultrasound monitoring, a clinician moves the emitter and detector around the area to be monitored to create an image. In the case of a fixed position, wearable monitor, the use of multiple probes or an array of probes may allow for the boundary of the hematoma to be monitored for growth. As more probes detect the presence of pooled blood, the algorithm can assess the risk of a growing hematoma based on rate and size. The transducer probes can be configured and attached to the patient in a similar fashion as described above for other modalities.

### Combinations of Modalities

Patients will typically begin to be ambulatory approximately four hours following the intervention. For the period between four hours and discharge (normally 24 hours), the patient will change orientation and have increased motion and thus introduce many factors that could lead to false signals or mask real signals. The use of an accelerometer, temperature sensors, O2 sensors, or other modalities may be used to understand the potential for changing signals in the sensing modality for bleeding risk. For instance, a change in impedance that corresponds with a signal from an accelerometer indicating the patient is now in a sitting position may be considered non-problematic, where if the patient was still in a horizontal position, bleeding may be alerted by the same change in impedance.

A combination of sensing modalities could be used to aid in a more predictive algorithm or to understand severity. For instance, blood pressure could be monitored to indicate a drop in blood pressure that might correspond with a retroperitoneal bleed. Or a strain gauge monitoring skin tension could be used as input to the algorithm to understand if a sub-dermal hematoma is leading to swelling. A photoplethysmogram (PPG) sensor could be used to continuously monitor capillary properties, such as blood pressure. A drop in blood pressure that corresponds to a significant drop in impedance may indicate a bleed that is severe enough to have systemic effects warranting a high level alert. Similarly, a combination of PPG sensors could be used to look for pressure changes along the length of the patch which could be an indication for a blockage in the underlying vessel that leads to a drop in tissue pressure distal to the blockage and an increase in pressure above the blockage.

### Algorithm for Detection and Threshold for Alerts

An algorithm of a computer program for detecting the development of a hematoma based on the data from the detector devices may provide alerts upon a threshold rate of change such volume or size of detected blood is envisioned. The algorithm may utilize multiple inputs, and process data in order to provide simple feedback to the patient or health care professional on the status of the access site. Most embodiments of the sensing modality and array will have the ability to interrogate the area of concern at different depths, locations and time points, and may optionally have the ability to interrogate on different frequencies to differentiate different structures and materials within the area of concern. Any or all of these inputs may be used to interpret the status of the access site.

A baseline measurement of the area directly following the surgery and sheath removal may be helpful to determine changes in detected blood without the inclusion of patient to patient variability or placement location variability. Optionally, a baseline measurement with the access sheath still in place after the therapeutic procedure is completed could also be used to calibrate the baseline with a known object (the sheath) in the field of sensing. Optionally, a baseline measurement could be taken over the course of several minutes following the removal of the sheath.

It is anticipated that most of the procedures will also involve a vascular access closure device to aid in vessel closure after the procedure. These devices could be metallic, polymeric, or biologic in nature, and may have the form factor of a fiber, patch, frame, clip, mesh or other configurations. The baseline calibration algorithm will need to have the ability to account for the presence of a variety of devices which could be at the access site in the vessel, outside the vessel, or in the access track. Since these devices are not anticipated to disappear, even in the case of an absorbable device, in the timeframe of concern, the ability to account for their presence will likely be done during the initial device calibration phase.

An exemplary wearable monitor system may be configured to operate a calibration wherein a first or first set of emitters and a first or first set of detectors are activated to determine a signal followed by different emitters and detectors or sets of emitters and detectors to determine which emitter or set of emitters provide a signal and measurement of to the area of interest, including the access track or tissue track, such as from the skin incision to the vessel puncture. The access track may extend along a line or a plane through the tissue and the emitters and detectors may be configured to detect this access track during the calibration or operation of the calibration algorithm. The initial calibration scheme may result in feedback that informs the algorithm on the controller on which combination of emitter devices and detector devices result in the best signal over the area of interest and therefore, which emitter devices and detector devices should be used over the course of monitoring. The use of an array of emitter and detector devices allows the correct positioning of the monitoring devices without the need for having to physically relocate the monitoring patch which would result in further variability and noise. The initial calibration procedure may also inform the algorithm which levels of current and/or which frequency to use for subsequent monitoring.

It is anticipated that the threshold for concern and alert may not be related to a single point in time measurement, but a trend over time. For example, a single point decrease in impedance may not indicate that there is presence of a hematoma and could be due to patient movement, change in skin hydration conditions, etc, but a steady decreasing trend over a period of time may indicate the growth of a hematoma. The location of this change may also be important, as a change in blood presence near the access site in the vessel in the shorter time points will likely precede a change in blood presence closer to the skin level, and may be weighted differently in the algorithm.

The algorithm will need to account for changes in the overall physiology of the patient, especially since the patient will be recovering from a surgery during the use of this device. This could include changes in blood pressure, pulse rate, blood oxygen level, temperature, hydration (including bulk and skin hydration level), and other potential factors. These factors could be accounted for by looking at overall trends that affect all of the sensors in an array in a similar way versus a subset of sensor readings changing. Alternatively, the previous discussion on combinations of sensing modalities may be used to account for overall physiological changes. Other sensing modalities could be in a location remote to the wearable monitor but could still be in communication with the algorithm and may be either wired, wirelessly, or through input from clinicians. For example, a smart watch monitoring O2 saturation, respiration rate or blood pressure could communicate back to the controller of the wearable hematoma monitor.

When the algorithm has detected changes that may indicate the presence of a bleeding event or hematoma, there could be a single threshold level for concern and alert, or there could be multiple levels. For a multiple level alert system, an initial ‘warning’ alert could prompt additional investigation from health care professionals to scrutinize the area with other detection modalities including ultrasound or manual palpation. This warning could be followed by an ‘urgent’ level that would indicate higher need to address issues at the access site immediately.

### Treatment Functionality

A wearable solution could have a means for treatment in addition to monitoring. Most often, a nurse will apply pressure over the wound to slow or stop bleeding when present. An inflatable cuff or a band with an inflatable pillow positioned over the wound could be combined with a sensor and programmed to apply pressure when an alert for a bleeding risk is identified. Another method could be an electrically activated plunger that draws power from the monitor. This could either be maintained until there are indications bleeding has stopped or just be used as a means until nursing can arrive for further evaluation. Other potential means to arrest bleeding could be repositioning the patient, applying other compression devices that are attached to the patient or the bed which they are positioned on, or applying clotting or hemostatic agents directly to the bleeding area. Another means of treatment might be to allow a window or open aperture through the monitoring patch which may allow the caretaker to apply pressure or otherwise inspect or treat the access track without having to reposition the monitoring patch. In exemplary embodiments, the emitter and detector devices are located on sides of the access track leaving the tissue directly over the access track available for an aperture in the monitoring patch.

### Other Monitoring Locations

These methods and modalities are described in detail for monitoring for bleeding complications following catheter interventions and specifically in the groin. These methods could also be employed to monitor for bleeding following jugular, brachial, radial or other vascular intervention sites. The wearable monitor system would also be useful in monitoring bleeding in areas of anastomosis or in chronic access sites such as in vascular access grafts for dialysis, in central venous or indwelling catheters or ports, etc. Another application could be hemodialysis fistula maturation where sensing the blood vessel diameter and also including alternating compression used to promote remodeling of the tissue can be envisioned.

In one possible embodiment, all of the functionality of the device is contained in one unit which is taken out of the package, powered on, applied to the area to be monitored, calibrated, and a baseline is collected. The device will then autonomously monitor against this baseline and be fully disposed of after it is no longer needed for monitoring.

A wearable monitor may be disposable or contain portions that are disposable, such as a monitoring patch of the device which will contact the patient's skin. The emitter and detector devices along with other powered components and the battery may be detachably attachable to the wearable monitor. In an exemplary embodiment a monitor patch, a pad or sheet containing the plurality of emitter and detector devices, is detachably attachable to a monitor control assembly that may include the controller, computer, battery, alert device and/or wireless transmitter. In this way, a monitor patch may be used for a patient and then detached and a new monitor patch may be attached to the monitor control assembly and applied to a second patient. This enables the wearable monitor system to be lower cost as many of the components are reused. Also, this arrangement of a detachable and monitor patch may prevent a requirement of sterilization or cleaning of the wearable monitor between patients. A signal connector may be configured between the monitor patch and the monitor control assembly for providing the signals from the emitter and detector devices to the controller and computer. The monitor control assembly may be removed from the monitor patch after the patient no longer needs monitoring and may then be recharged in between uses and, optionally, the software upgraded in order to maintain the latest functionality.

The emitter and detector devices may operate under low voltage power, such as about 3 volts or less and may be electrically isolated from the patient, and have a material covering that is water resistant to prevent damage or shorting of the system during the in-use period.

The wearable monitor system may be controlled by a controller that may include a computer or microprocessor that receives the detection signals and initiates running calibration and interrogation of the tissue. A controller may include output to switches and other controllers of the system.

### Definitions

A transducers, as used herein, is a device that converts one form of energy into another or input energy from one medium to another. Transducers are commonly used in sensing and measurement applications, transducers can transform signals such as mechanical energy into electrical signals. An electrode is one form of transducer as used herein, wherein an electrode, an electrically conductive contact receives electrical power or signals from the lead lines of the wearable device can input an electrical signal into tissue and/or detect or receive an electrical signal from the tissue.

A four-point probe, as used herein, is a configuration of electrodes that enables 4-wire sensing, wherein an input electrode emitter and output electrode emitter are configured outside of a respective input electrode detector and output electrode detector, wherein the input electrode detector and output electrode detector are between the two electrode emitters. An electrical signal, including an electrical current is input into the tissue by the input electrode emitter and passed through the tissue to the output electrode emitter and the electrical signal is measured between the input and output electrode detectors, such as the voltage drop across the input and output electrode detectors. The electrical signal includes current and voltage and may be alternating or provided at various frequencies for the measurement. The separation of current and voltage electrodes eliminates the lead and contact resistance from the measurement. This is an advantage for precise measurement of low resistance values.

An electrode is an electrically conductive contact that receives and transfers, received or inputs, an electrical signal into the tissue from the wearable monitor and may be a metallic layer of a monitoring device or monitoring array and may be discrete, such a small circle or ring having a diameter or maximum length across the electrode surface of about 50 mm or less, about 35 mm or less, about 25 mm or less, about 15 mm or less and any range between and including the values provided.

The summary of the invention is provided as a general introduction to some of the embodiments of the invention and is not intended to be limiting. Additional example embodiments including variations and alternative configurations of the invention are provided herein.

### BRIEF DESCRIPTION OF SEVERAL VIEWS OF THE DRAWINGS

The accompanying drawings are included to provide a further understanding of the invention and are incorporated in and constitute a part of this specification, illustrate embodiments of the invention, and together with the description serve to explain the principles of the invention.

FIG. 1 shows a cross-sectional view tissue and a vessel with an introducer extending through the tissue and into a vessel with a catheter extending through the introducer and into the vessel.

FIG. 2 shows a cross-sectional view of the tissue and vessel shown in FIG. 1 with the introducer and catheter extracted and a hematoma forming from the vessel puncture.

FIG. 3 shows a cross-sectional view of the tissue and vessel shown in FIG. 2 with hematoma growing in size.

FIG. 4 shows a cross-sectional view of the tissue and vessel shown in FIG. 2 with hematoma growing in size and causing swelling of the tissue.

FIG. 5 shows a diagram of a femoral artery access location for a vascular catheterization with a hematoma forming from the vessel puncture.

FIG. 6 shows a skin interface side or interfaceside of wearable monitor system having an array of emitter devices and detector devices configured on a monitor patch and a pair of straps extend from the wearable monitor to secure the wearable monitor to a person.

FIG. 7 shows a side view of a wearable monitor system having an array of emitter devices and detector devices configured on a monitor patch and a pair of straps extend from the wearable monitor to secure the wearable monitor to a person and including a computer, controller, alert device, battery and wireless transmitter.

FIG. 8 shows an end view of a wearable monitor system having an array of emitter devices and detector devices configured on a monitor patch and a pair of straps extend from the wearable monitor to secure the wearable monitor to a person and including a computer, controller, alert device, battery and wireless transmitter.

FIG. 9 shows a diagram of a femoral artery access location for a vascular catheterization with a hematoma forming from the vessel puncture and a wearable monitor system configured thereover to detect the bleeding event and monitor the hematoma.

FIG. 10 shows a diagram of an emitter device and detector device array having some of the emitter devices activated to produce an emitter input into tissue and an array of detector devices activated to measure the emitter input.

FIG. 11 shows a diagram of a wearable monitor system having an emitter device array configured in a V-shape with a first set of emitter devices located near to or on a first side of the skin incision and having a small offset distance and a plurality of pairs of emitter devices with progressively increasing offset distances between them configured along the V-shape and extending away from the vertex of the V-shape and skin incision, and an array of detector devices configured between the pairs of emitter devices.

FIG. 12 shows a cross-sectional view of tissue with an array of emitter devices as shown in FIG. 11 to produce a range of depths of measurement.

FIG. 13 shows a cross-sectional view of tissue and a vessel with an array of emitter devices to produce a range of depths of measurement to determine the size and location of a hematoma.

FIG. 14 shows a cross-sectional view of tissue and a vessel with array of emitter devices a to produce a range of depths of measurement to determine the size and location of a hematoma and detector devices configured between the emitter devices.

FIG. 15 shows a diagram of a wearable monitor system having an electrode emitter array configured in a V-shape with a first set of emitter devices configured close to the skin incision having a small offset distance and a plurality of pairs of electrode emitter with progressively increasing offset distances away from the skin incision and an array of electrode detectors configured between the pairs of electrode emitters.

FIG. 16 shows a cross-sectional view of tissue with an array of electrode emitters as shown in FIG. 15 to produce a range of depths of measurement.

FIG. 17 shows a diagram of a wearable monitor system having an ultrasound emitter array configured in a V-shape with a first set of ultrasound emitters configured close to the skin incision having a small offset distance and a plurality of pairs of ultrasound emitter with progressively increasing offset distances away from the skin incision and an array of ultrasound detector devices configured between the pairs of ultrasound emitters.

FIG. 18 shows a cross-sectional view of tissue with an array of ultrasound emitters as shown in FIG. 17 to produce a range of depths of measurement.

FIG. 19 shows a diagram of a wearable monitor system having a Near IR emitter array configured in a V-shape with a first set of Near IR emitters configured close to the skin incision having a small offset distance and a plurality of pairs of Near IR emitter with progressively increasing offset distances away from the skin incision and an array of Near IR detector devices configured between the pairs of Near IR emitters.

FIG. 20 shows a cross sectional view of tissue with an array of Near IR emitters as shown in FIG. 19 to produce a range of depths of measurement.

FIG. 21 shows a wearable monitoring patch configured with eight electrodes arranged in an array useful for four-point impedance monitoring.

FIG. 22, FIG. 23, FIG. 24 and FIG. 25 each show a wearable monitor having an open area configured to extend over a skin incision and access track and a plurality of transducers configured around the open area to produce interrogation axes that extend along the length axis or substantially parallel thereto within about 20 degrees and across the length axis.

FIG. 26, FIG. 27 and FIG. 28 each show an array of transducers, that may be emitter devices or detector devices or activated to function as either an emitter device or detector device to monitor the progression of a bleed over time. The shaded points represent emitters and/or detectors which are active, illustrating that the emitters and detectors can be activated or deactivated to follow a bleed as it progresses in multiple directions.

FIG. 29, FIG. 30, FIG. 31 and FIG. 32 each show a monitoring array with a plurality of emitters and detectors configured on opposing sides of a blood vessel and used for bioimpedance measurements, and a respective graph of bioimpedance versus a baseline impedance.

FIG. 33 shows a perspective view of a monitoring array including a plurality of transducers with two emitters and two detectors activated bioimpedance measurements.

FIG. 34 shows a perspective view of a monitoring array shown in FIG. 33 now with two different emitters and two different detectors activated to enable a greater depth of monitoring in the tissue.

FIG. 35 shows an exemplary wearable monitor having a monitoring array that has a monitor patch with an open area for accessing the skin incision and/or access track or vessel puncture and has leads that extend to monitoring hubs configured around the vessel access site, wherein each monitoring hub has an emitter and a detector.

FIG. 36 shows the exemplary wearable monitor shown in FIG. 35 with bold arrows extending between the monitoring hubs to show the different regions of tissue that can be monitored between the monitoring hubs.

FIG. 37 shows a graphical representation of response signals that may be achieved with the wearable monitor shown in FIG. 35, wherein in FIG. 37 the response, such as impedance is similar for each of the four perimeter monitoring paths between the monitoring hubs and also for the two traversing monitoring paths across the length axis of the wearable monitor.

FIG. 38 shows a graphical representation of the response signals wherein the two traversing monitoring paths across the length axis of the wearable monitor have significantly different values than the four perimeter monitoring paths between the monitoring hubs indicating a bleeding event, wherein the larger width lines may indicate a drop in impedance.

Corresponding reference characters indicate corresponding parts throughout the several views of the figures. The figures represent an illustration of some of the embodiments of the present invention and are not to be construed as limiting the scope of the invention in any manner. Some of the figures may not show all of the features and components of the invention for ease of illustration, but it is to be understood that where possible, features and components from one figure may be included in the other figures. Further, the figures are not necessarily to scale, some features may be exaggerated to show details of particular components. Therefore, specific structural and functional details disclosed herein are not to be interpreted as limiting, but merely as a representative basis for teaching one skilled in the art to employ the present invention.

### DETAILED DESCRIPTION OF THE ILLUSTRATED EMBODIMENTS

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Certain exemplary embodiments of the present invention are described herein and are illustrated in the accompanying figures. The embodiments described are only for purposes of illustrating the present invention and should not be interpreted as limiting the scope of the invention. Other embodiments of the invention, and certain modifications, combinations, and improvements of the described embodiments, will occur to those skilled in the art and all such alternate embodiments, combinations, modifications, improvements are within the scope of the present invention.

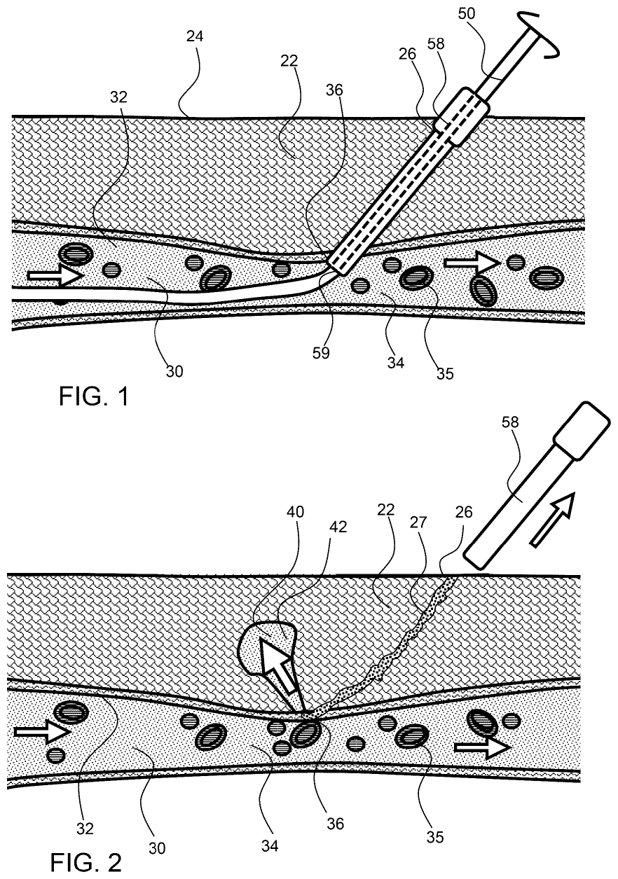

Referring now to FIGS. 1 to 4, an introducer 58 is inserted through a skin incision 26 in the skin surface 24 and extends through the tissue 22 and into a vessel 30 with a catheter 50 extending through the introducer and into the vessel to produce a vessel puncture 36 in the vessel wall 32. As shown in FIG. 1, the distal end 59 of the introducer extends into the vessel 30 and the catheter extends into the vessel through the introducer. Vessel blood 34 with platelets 35 flow in the vessel, which may be a vascular vessel such as a vein or artery.

As shown in FIG. 2, the introducer 58 and catheter 50 are extracted from the tissue 22 and the vessel 30 leaving an access track 27 in the tissue 22. A hematoma 40 is forming from the vessel puncture 36 with blood 42 pooling in the tissue 22.

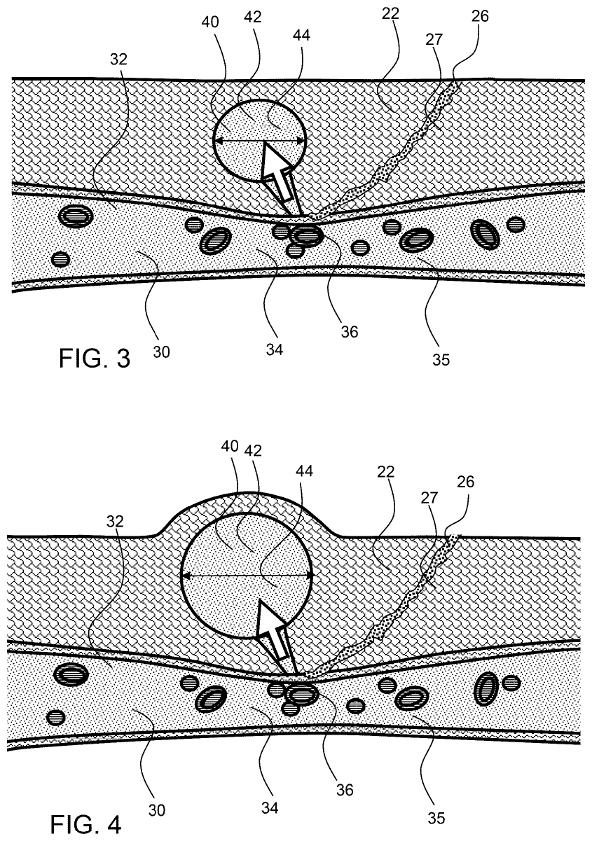

As shown in FIG. 3, the blood 42 continues to pool and the hematoma is enlarged with a hematoma length 44 from one side to an opposing side. As shown in FIG. 4, the hematoma is enlarged further and the hematoma length has increased from the length of the hematoma shown in FIG. 3. The tissue 22 then exhibits swelling due to the pooling blood as shown in FIG. 4.

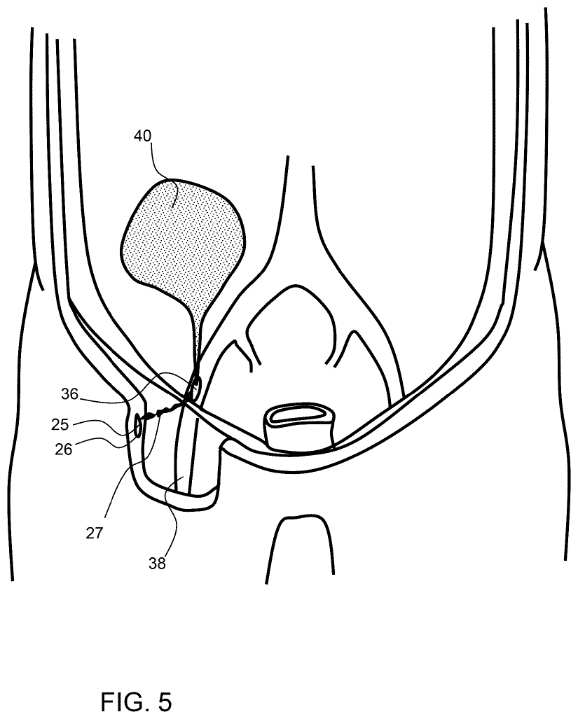

As shown in FIG. 5, a venous access site 25 is in a femoral artery 38 and the vessel puncture 36 has resulted in a hematoma. The venous access has a skin incision 26, an access track 27 and a vessel puncture.

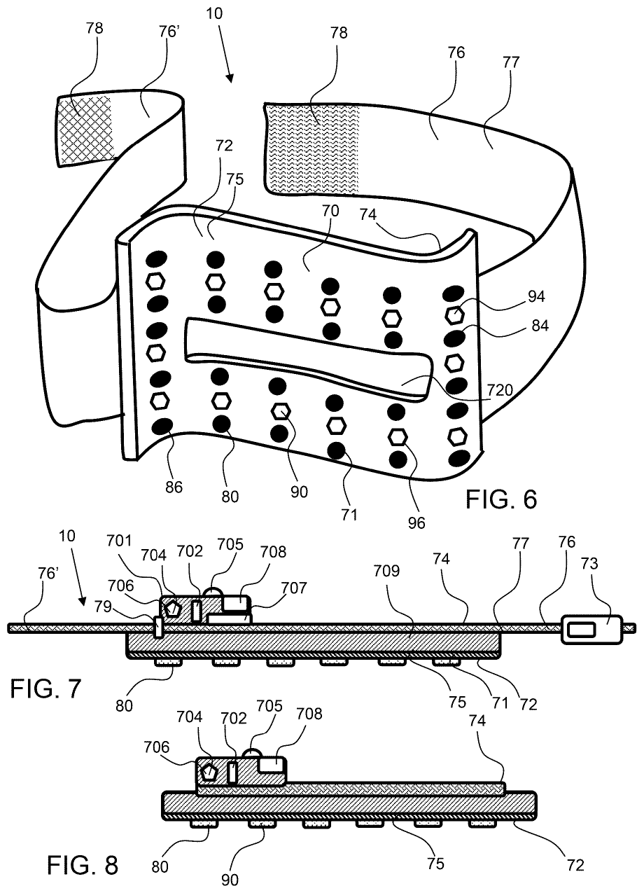

Referring now to FIGS. 6 to 8, a wearable monitor system 10 includes a wearable monitor 70 configured to be placed over tissue of a person such as over an access track to a blood vessel. The wearable monitor may be placed over a venous access site to detect a bleeding event that may result in a hematoma. The wearable monitor 70 has a plurality of transducers, that are configured to switch from an emitter device 80 to a detector device 90 to detect changes in the tissue that can be correlated to a bleeding event, such as a hematoma, from the blood vessel. The wearable monitor has an interface side 72 configured with a monitoring array 71 including a plurality of emitter devices 80 and a plurality of detector devices 90. The monitoring array may be configured on a monitoring patch 75 that is a sheet of material that may be supple and flexible as described herein. The wearable monitor may have an open area 720 that is configured to extend over an access location, such as a venous access site and may extend over a skin incision, an access track and vessel punction location. The monitoring array and the interface side of the monitoring patch may be planar when configured on a planar surface but may conform to contours of a person's body for effective contact with the skin for diagnostics. As shown, the monitoring array has emitter rows 84 and emitter columns 86 and detector rows 94 and detector columns 96. One or more of the emitter devices 80 may be and one of more of the detector devices 90 may be activated by the controller 704 for interrogation of the tissue for a bleeding event such as a hematoma. The emitter devices and detector devices are coupled to a monitor patch 75 that may be a planar material configured to retain the emitters and detectors in a relatively fixed position with respect to each other. As described herein, the monitor patch 75 may be a fabric or a polymeric material such as silicone or urethane, and may be non-conductive and elastic to avoid interference with the emitter input and detection of the emitter input by the detector devices.

An alternate attachment component 77, such as straps 76, 76′ may extend from the wearable monitor 70 to enable the wearable monitor to be secured on a person's skin over a venous access location. As shown, the straps 76, 76′ have respective strap fasteners 78, 78′ such as hook and loop fastener, to couple the straps together around a person. Alternatively, an attachment component 77 may include an adhesive surface that adheres with contact with the skin to affix the wearable patch to the skin for the duration of monitoring.

As shown in FIGS. 7 and 8, a wearable monitor system 10 has a wearable monitor 70 with a controller 704 that may include a computer 706, or microprocessor alert device 705, battery 702 and wireless transmitter 708 configured thereon, on the exposed side 74. As described herein, the computer, and/or the alert device may be configured in a remote location, not configured on the wearable monitor, and communication by the wireless transmitter 708 may provide data from the detector devices 90 to the computer and instructions from the computer to the controller.

A monitor patch 75, a pad or sheet containing the plurality of emitter and detector devices or monitoring array 71, is detachably attachable to a monitor control assembly 701 that may include the controller 704, computer 706, battery 702, alert device 705, pump 707 and/or wireless transmitter 708. In this way, a monitor patch may be used for a patient and then detached, and a new monitor patch may be attached to the monitor control assembly and applied to a second patient. This enables the wearable monitor system to be lower cost as many of the components are reused. Also, this arrangement of a detachable and monitor patch may prevent a requirement of sterilization or cleaning of the wearable monitor between patients. A signal connector 79 may be configured between the monitor patch and the monitor control assembly for providing the signals from the emitter and detector devices to the controller and computer. Also, the signal connector may provide power from a power source, such as a battery 702 to the emitter devices 80 and/or detector devices 90. The monitor control assembly may be removed from the monitor patch after the patient no longer needs monitoring and may then be recharged in between uses and, optionally, the software upgraded in order to maintain the latest functionality.

As shown in FIG. 7, an attachment component 77 may include a means to tighten or expand the wearable monitor 70 to apply pressure over a skin incision, access track and/or vessel puncture to reduce a bleeding event. When a bleeding event is detected, the controller 704 may initiate a command to increase pressure by inflating a bladder 709 via a pump 707, wherein the bladder may be configured with the monitor patch 75 and the pump configured with the monitor control assembly 701. Also, a strap 76 may include a tightening mechanism 73, that pulls the strap to increase pressure applied by the monitor patch 75.

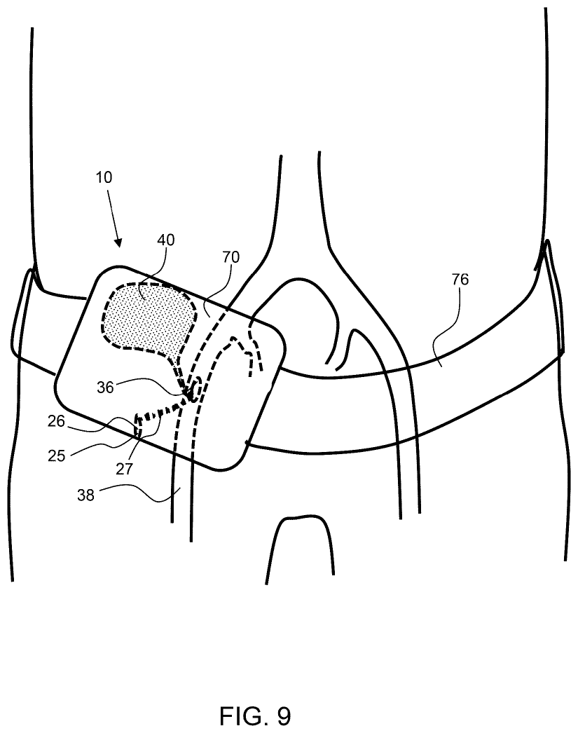

As shown in FIG. 9, an exemplary wearable monitor system 10 is configured over a venous access site 25 to detect a bleeding event, forming a hematoma. The wearable monitor 70 is attached over the venous access site 25 and extends from the skin incision 26 over the access track 27 and over the vessel puncture 36. The array of emitter and detector devices, as shown in FIG. 6, may be activated to detect a change and/or rate of change in the tissue, such as a pooling of blood.

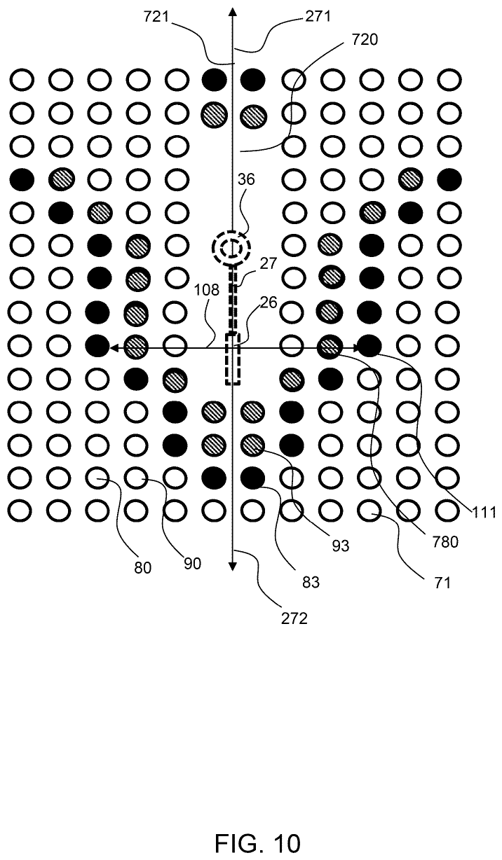

Referring now to FIGS. 10 to 14, specific emitter devices 80 and detector devices 90 may be activated for effective interrogation of the tissue and detection of a bleeding event, a change in the tissue. As shown in FIG. 10, the monitoring array 71 has a plurality of activated emitter devices 83 and activated detector devices 93. As described herein, a controller may activate a first array of activated emitter devices 83 and activated detector devices 93 and then activate a second array of activated emitter devices and activated detector devices. As shown, the activated emitter devices 83 and activated detector devices 93 form a respective V-shape around an open area 720 of the monitoring array 71 or wearable monitor. The V-shape expands from the vertex of the V-shape around the open area 720 and around the access location including the skin incision 26, access track and vessel puncture 36. The length axis 721 of the wearable monitor may be aligned with the access track axis 271 as shown and this may be a preferred orientation. Note that a set of activated emitter devices 83 and activated detector devices 93 may be electrodes 111 and activated to form a four-point probe 780 having an interrogation axis 108 across the length axis 721 or access track plane 272 and that separate pairs of activated emitter devices 83 and activated detector devices 93 may be turned on and off to interrogate along the length axis and along the access track. The length axis of the wearable monitor 70 may be configured to extend along an open area 720 and may be centrally located along or over an open area and again, may align with an access track for effective interrogation along the access track for a bleeding event.

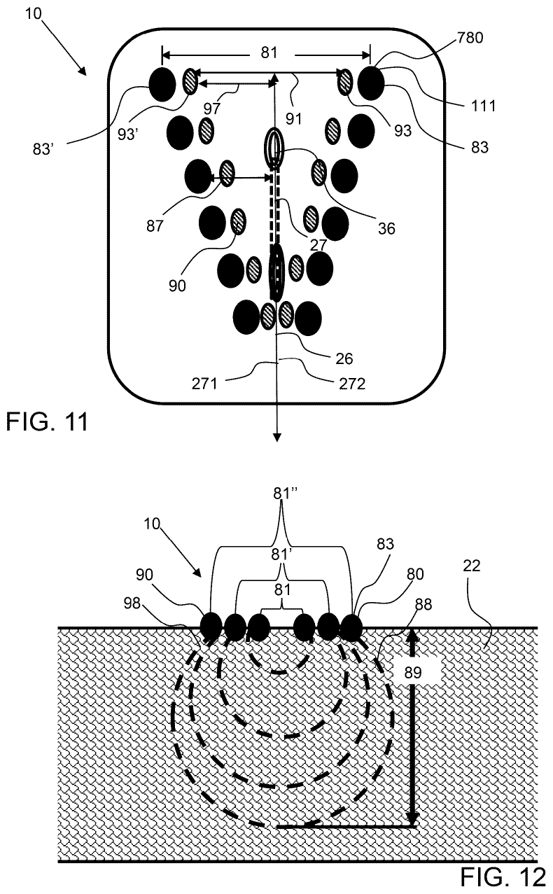

As shown in FIG. 11, the activated emitter devices 83 and activated detector devices 93 form a V-shape over the access track 27, with a plurality of activated emitter devices 83 on opposing sides of the access track and a plurality of activated detector devices on opposing sides of the access track 27. A pair of activated emitter devices 83, 83′ have an emitter offset distance 81 between them and for interrogation purposes, a different pair of activated emitter devices may be activated to change this emitter offset distance. Emitter devices that are further apart or have a greater emitter offset distance may measurement of tissue characteristics that are deeper. Likewise, the activated detector devices 93, 93′ have a detector offset distance 91 that may also be changed by the controller activating different detector devices and again, changing the detector devices may provide a more complete and accurate detection of changes in the tissue. Again, for interrogation purposes only a single pair of activated emitter devices and activated detector devices may be activated at a time for better resolution of interrogation. A plurality of activated devices is shown for the purposes of showing the V-shape over the access track. Again, each of the activated devices may be an electrode 111 and a pair of activated emitter devices 83 and activated detector devices may form a four-point probe 780.

As shown in FIG. 11, the access track 27 extends along an access track axis 271 that extends from the skin incision 26 to the vessel puncture 36 and forms an access track plane 272 along this access track that is generally orthogonal to the plane of the skin and extends between the skin incision and vessel puncture. As described herein, a controller and computer (not shown) may run a calibration to determine the location of the access track in the tissue and this may be accomplished by activating an emitter and/or detector device or devices with a first emitter track plane offset distance 87 and first detector track plane offset distance 97. The distance of the activated emitters and detectors from the access track plane 272 may be changed to effectively determine which emitters and detectors are most effective in monitoring the access track or tissue around the access track for a bleeding event. As shown, one or more emitters configured close to the skin incision 26 may be activated with a smaller emitter track plane offset distance than emitters closer to the vessel puncture 36, as the vessel puncture is deeper in the tissue and therefore may require a deeper input signal for effective monitoring for changes.

As shown in FIG. 12, a plurality of activated emitter devices 83 produce an emitter input 88 into the tissue 22 having an emitter input depth 89 in the tissue 22 that may be greater for emitters that are further apart from each other, having a greater emitter offset distance 81, 81′, 81″, or further away from a detector 90 as shown in FIG. 12. The emitter input passes through the tissue and is detected by the detector device 90 as a detection signal 98. As shown, three activated emitters devices 83 produce detections signals with different emitter input depths 89.

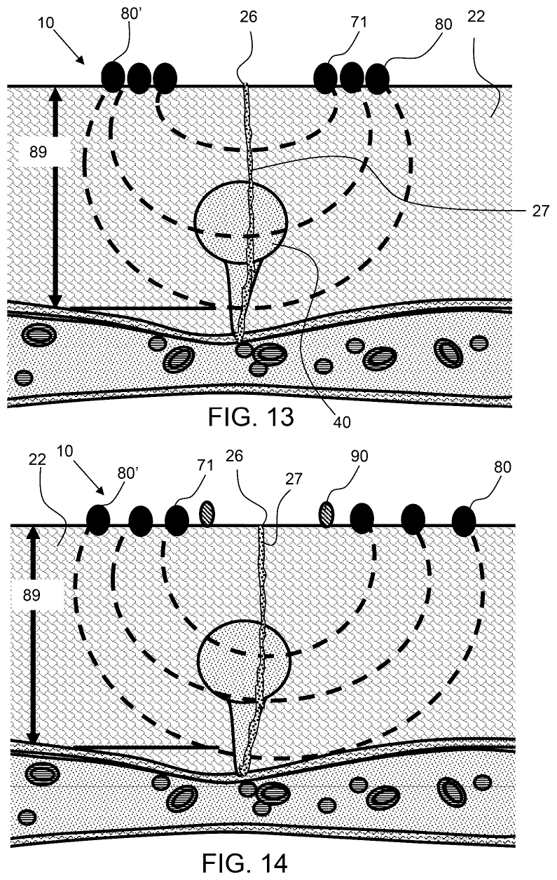

As shown in FIGS. 13 and 14, a monitoring array 71 is configured to interrogate tissue 22 to detect a bleeding event, such as a hematoma 40. As shown, the emitter devices 80, 80′ are configured on either side of the access track 27. As described herein, a controller may activate emitter devices to turn on in sequence and a detector device in sequence as well to provide data throughout the depth of the tissue. This type of sequencing of emitter and detectors may enable better detection of a bleeding event.

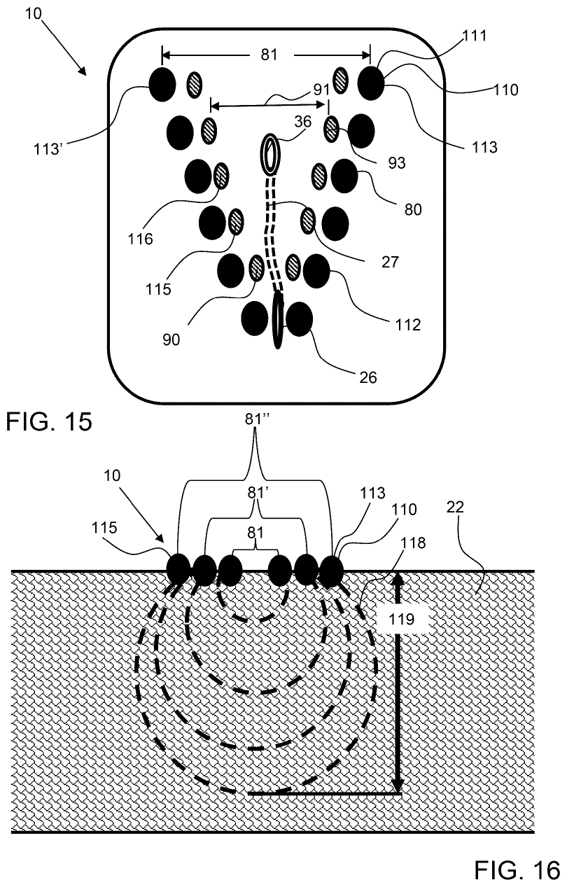

Referring now to FIGS. 15 and 16, the emitter devices 80 are electrode emitters 110 and the detector devices 90 are electrode detectors 115. An array of electrodes 112 may enable a controller to activate one or more of the electrodes 111 in the emitter array as an activated electrode emitter 113 and one or more of the electrodes 111 as an activated electrode detector 115. In this way, the system may turn on and off the electrodes as emitter and detectors to obtain data about the tissue along and around the access track 27. As shown, the activated electrode emitters 113 and activated electrode detectors 116 form a V-shape over the access track 27, with a plurality of activated electrode emitters 113 on opposing sides of the access track and a plurality of electrode detectors 116 on opposing sides of the access track 27. The activated electrode emitters are configured an emitter offset distance 81 and this distance may be changed by activating different electrode emitters. Electrode emitters that are further apart or have a greater emitter offset distance may provide measurement of tissue characteristics that are deeper. Likewise, the activated electrode detectors 116 have a detector offset distance 91 that may also be changed by the controller activating different detector devices and again, changing the detector devices may provide a more complete and accurate detection of changes in the tissue.

As shown in FIG. 16, a plurality of activated electrode emitters 113 produce an emitter input 118 into the tissue 22, an electromagnetic input including current at a voltage that may be alternating or input as a wave or oscillating input having a frequency and amplitude of voltage and/or current. The depth 119 of the emitter input into the tissue 22 may be greater for emitters that are further apart or further away from a detector. As shown the six activated electrode emitters have three different emitter offset distances 81, 81′ and 81″.

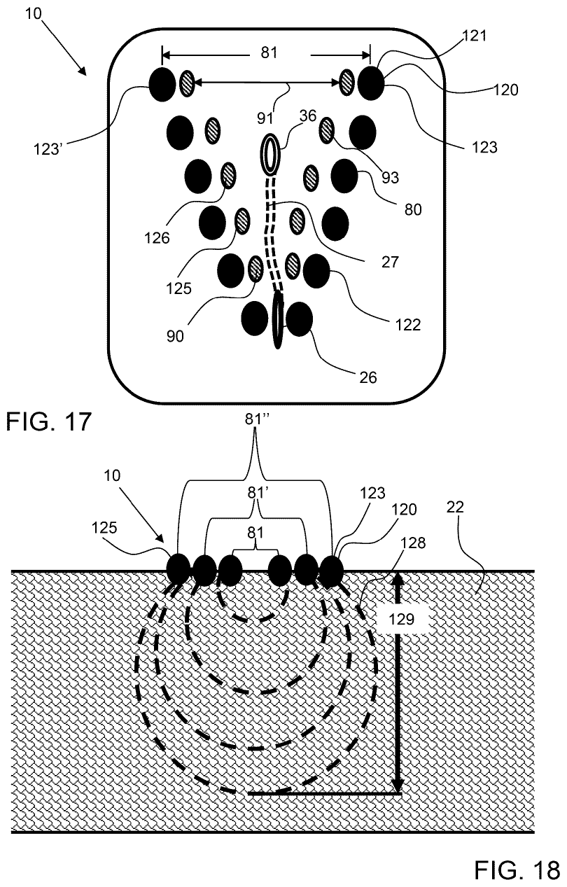

Referring now to FIGS. 17 and 18, the emitter devices 80 are ultrasound emitters 120 and the detector devices 90 are ultrasound detectors 125. An ultrasound emitter may be a sound wave emitter 121. An array of ultrasound emitters 122 may enable a controller to activate one or more of the ultrasound emitters 120 in the emitter array as an activated ultrasound emitter 123, and one or more of the ultrasound detectors 125 as an activated ultrasound detector 126. In this way, the system may turn on and off the ultrasound emitters 120 and ultrasound detectors 125 to obtain data about the tissue along and around the access track 27. As shown, the activated ultrasound emitters 123 and activated ultrasound detectors 126 form a V-shape over the access track 27, with a plurality of activated ultrasound emitters 123, 123′ on opposing sides of the access track and a plurality of ultrasound detectors 126 on opposing sides of the access track 27. The activated ultrasound emitters are configured an emitter offset distance 81 and this distance may be changed by activating different ultrasound emitters. Ultrasound emitters that are further apart or have a greater emitter offset distance may provide measurement of tissue characteristics that are deeper in the tissue. Likewise, the activated ultrasound detectors 126 have a detector offset distance 91 that may also be changed by the controller activating different detector devices and again, changing the detector devices may provide a more complete and accurate detection of changes in the tissue.

As shown in FIG. 18, a plurality of activated ultrasound emitters 123 produce an emitter input 128 into the tissue 22, a sound wave input including that may have an ultrasound frequency and amplitude. The depth 129 of the emitter input into the tissue 22 may be greater for emitters that are further apart or further away from a detector. As shown the six activated ultrasound emitters have three different emitter offset distances 81, 81′ and 81″.

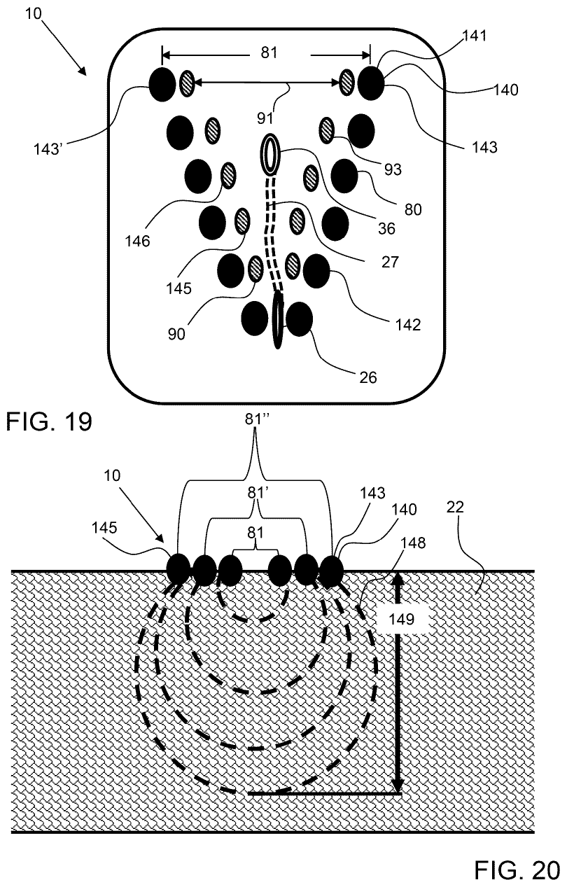

Referring now to FIGS. 19 and 20, the emitter devices 80 are Near IR emitters 140 and the detector devices 90 are Near IR detectors 145. A Near IR emitter may be an electromagnetic radiation emitter 141. An array of Near IR emitter 142 may enable a controller to activate one or more of the Near IR emitter 140 in the emitter array as an activated Near IR emitter 143 and likewise an array of Near IR detectors may enable a controller to activate one or more of the Near IR detectors 145 as an activated Near IR detectors 146. In this way, the system may turn on and off the Near IR emitters and detectors to obtain data about the tissue along and around the access track 27. As shown, the activated Near IR emitters 143 and activated Near IR detectors 146 form a V-shape over the access track 27, with a plurality of activated Near IR emitters 143 on opposing sides of the access track and a plurality of Near IR detectors on opposing sides of the access track 27. The activated Near IR emitters are configured an emitter offset distance 81 and this distance may be changed by activating different Near IR emitters. Near IR emitters that are further apart or have a greater emitter offset distance may provide measurement of tissue characteristics that are deeper. Likewise, the activated Near IR detectors 146 have a detector offset distance 91 that may also be changed by the controller activating different detector devices and again, changing the detector devices may provide a more complete and accurate detection of changes in the tissue.

As shown in FIG. 20, a plurality of activated Near IR emitters 143 produce an emitter input 148 into the tissue 22, emits NIR light into the tissue as the emitter input and a NIR detector device detects a detection signal of NIR light that has passed through the tissue from the one or more NIR emitter devices. NIR which may be an electromagnetic input including such as an electromagnetic radiation emitter. Again, a NIR emitter may utilize light as an input. The depth 149 of the emitter input into the tissue 22 may be greater for emitters that are further apart or further away from a detector. As shown, the six activated Near IR emitters have three different emitter offset distances 81, 81′ and 81″.

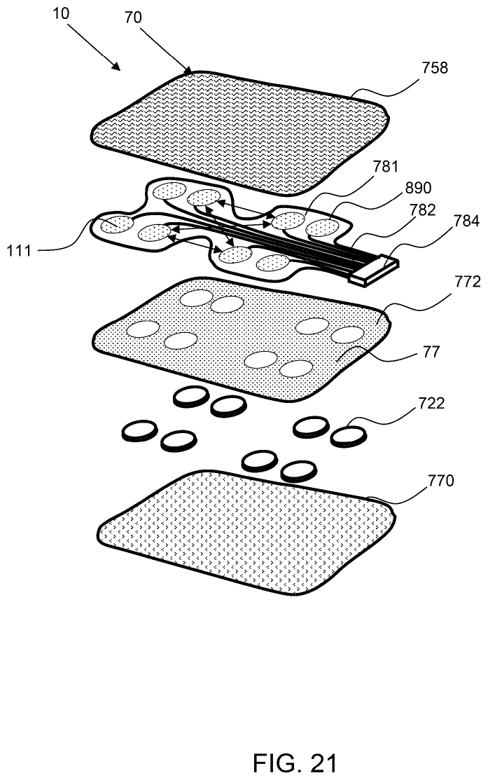

Referring now to FIG. 21, a wearable monitor 70 is shown that is particularly suited for electrical impedance tomography, where the emitter and detector devices are skin adherent electrodes that can emit and receive current and/or monitor for voltage drops. The electrodes 111 have electrical leads 782 that extend from a power source to the transducer 890, or emitter device or detector device. The electrical leads may extend to a connector or lead hub 784 that may be connected for retrieving data, charging the power source, coupling with a controller and the like. As shown the wearable monitor has conductive patches 722, that may be a portion of the electrode 111 and the patches may extend through apertures in the skin adhesive layer 772, the attachment component 77 for the wearable monitor 70. A circuit layer 781 includes the electrical leads 782 and a lead hub 784 for the electrical leads, as well as the monitoring devices that are coupled to the conductive patches 720. A cover layer 758 extends over the circuit layer 781 and may protect the circuit layer from contact and exposure to liquids that might affect the detection signal. A release layer 770 may be configured over the adhesive layer 772 and may be removed prior to application to a person's skin for monitoring.

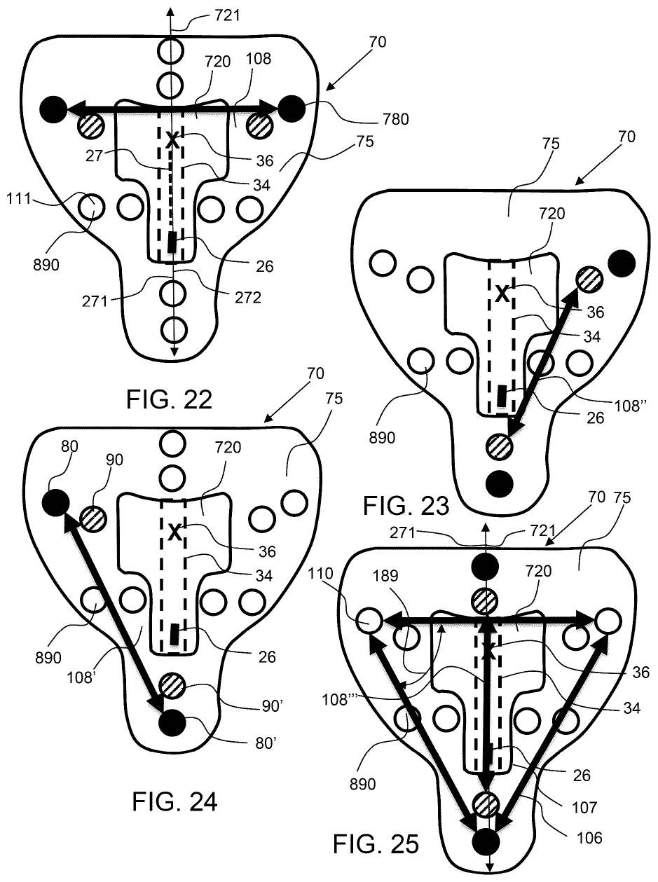

Referring now to FIGS. 22 to 25, a wearable monitor 70 of a wearable monitor system has an irregular shape configured to conform around a vessel access such as a femoral access catheter procedure access site, having an open area 720 to enable access to the skin incision 26 and monitor the skin along the access track 27 to the vessel puncture 36. This open area may be accessed for the application of gauze or to enable inspection for infection. Note that the monitor patch 75 extends around the perimeter of the open area 720, forming a window open area, an opening within the monitor patch. The open area 720 preferably extends over the entirety of the access track from the skin incision to the vessel puncture location, which is at the highest risk for a hematoma. This open area allows visualization and manual palpations that are typically used to screen for hematomas. Also shown in FIG. 22 are four different combinations of emitter and detector devices that result in four different four-point probes 780 each having a different interrogation axis 108, 108′, 108″, 108″′ and may have different depths of tissue interrogation. There are more combinations possible that are not shown. A controller may activate a first four-point probe measurement and then deactivate this first four-point probe and then activate a second four-point probe. Note that transducers 890, electrodes 111 may be shared for the formation of different four-point probes with different partnering pairs of transducers to form different integration axes. The data from multiple four-point probes with different interrogations axes may more effectively and more quickly identify or detect a bleeding event and may more effectively confirm the growth rate and location of the bleeding in the tissue. Note that the transducers 890 may be activated as emitter devices or detector devices.

As shown in FIG. 25, a pair of transducers are activated and configured along the length axis 721 of the wearable monitor 70 which is configured to align with the access track axis 271 when applied to the skin over or around the access track, as shown. These pairs of transducers on opposing sides of the open area 720 along the length axis 721 form a four-point probe 780 with an interrogation axis 108′″ that extends along or parallel with the length axis and access track 27. This configuration enables interrogation along an interrogation axis 108″′ for changes in the tissue along the access track, including changes in impedance along the blood vessel 34 or access track 27, where a bleeding event is most likely.

As shown in FIG. 25, the interrogation axes shown may form an interrogation perimeter 106 around the monitored area 107, which includes the open area 720 and the access site including the skin incision, access track and vessel puncture. An interrogation perimeter is a perimeter formed by the intersection and extensions of intersecting interrogation axes to form said perimeter around an area of the wearable monitor 70. This ensures that any bleeding events that may propagate in any direction from the access site will be detected. Also shown in FIG. 25, an axis offset angle 189 between interrogation axes is shown and in this case in shown wherein the two adjacent interrogation axes share at least one transducer, in this case, an emitter transducer or electrode emitter 110. The axis offset angle is the included angle between the two interrogation axes from an intersection of the two interrogation axes. An axis offset angle 189 may be orthogonal such as when a monitoring array employs a grid of transducers that are configured in parallel rows, whereby an interrogation axis may extend along a row, or between or across rows to form orthogonal axis offset angles.

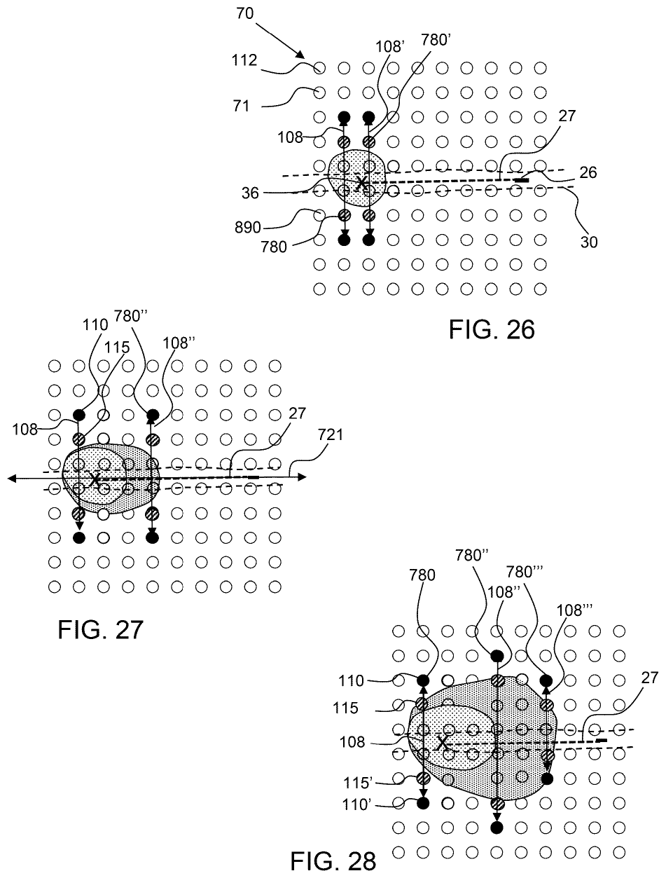

Referring now to FIGS. 26 to 28, an array of electrodes 112 on a wearable monitor 70 may be transducers 890 that can be configured as an electrode emitter 110 or electrode detector 115. As shown, the black electrodes illustrate activated electrode emitters 110 configured to emit an emitter input into the tissue for detection by one or more electrode detectors, illustrated with cross-hatching, to detect a location of a bleeding event below the skin. As the bleed progresses in size and location from FIG. 26 to FIG. 28, additional electrodes aligned with the outline of the bleed may be activated by the controller, while other electrodes which are no longer at the edge of the bleed boundary may be deactivated to ensure that an accurate representation of growth is captured while optimizing the power usage of the device. The controller may automatically activate electrodes adjacent electrodes that have detected a drop in impedance and in this way, the bleeding event growth can be effectively mapped or monitored. The controller may control electrodes to activate in a sequence to produce interrogation axes along a bleeding event or across the access track 27, and/or across the length axis 721 of the wearable monitor to determine the size and/or location of the bleed. The controller may sequence electrodes to monitor across an access track until the monitored change in impedance is below the threshold value indicating a bleed in that interrogation axis. As shown, in FIG. 26, two four-point probes 780, 780′ are configured to produce interrogation axes across the access track and the two four-point probes are adjacent each other with respect to the transducer array, monitoring array 71 or array of electrodes 112. A small bleed as indicated by the dotted area is detected by the wearable monitor 70 in FIG. 26.

As shown in FIG. 27, one of the four-point probes from FIG. 26 is utilized, however the controller has activated a second four-point probe 780″ that is further offset from the vessel puncture along the access track 27, or offset along the length axis 721, and has an interrogation axis 108″, that is activated to determine how much the bleeding has progressed. Note that the controller may activate four-point probes across the access track or along the length axis in an iterative or sequenced manner to determine the size and progression of the bleeding event. A controller may automatically activate adjacent transducers to form adjacent four-point probes, which may have a parallel interrogation axes. An adjacent second four-point probe to a first four-point probe having a detection signal below a threshold, indicating a bleeding event, may be automatically activated to determine how far the bleeding event extends in the tissue. Successive adjacent four-point probes which may have parallel interrogation axes may be activated until one or more has a detection signal above the threshold value.

As shown in FIG. 28, three four-point probes 780, 780″ and 780″′ having a respective interrogation axis 108, 108″ and 108″′ are used to determine the extent of the bleeding event. In FIG. 28, a four-point probe 780″ between the outer two four-point probes, or more centrally located along the access track 27 between the skin incision and the vessel puncture has a greater emitter offset distance, or distance between the two electrode emitters to enable an interrogation deeper into the tissue than the other two four-point probes.

Note that not all four-point probes may be activated at the same time. For better resolution of detection, only one four-point probe may be activated at a time and there may be a small delay time between each monitoring event having a four-point probe activated. Also note that four-point probes that extend on a side of the access track 27 may be employed, wherein the two electrode emitter and two electrode detectors are all configured on the same side of the access track and may be substantially parallel with the access track axis, or within about 20 degrees of parallel, wherein the access track axis in a line from the skin incision to the vessel puncture.

As shown, the activated electrodes may form four-point probes with an interrogation axis that extends across the bleeding region in the tissue. FIG. 26 to FIG. 28 show the progression of the bleeding event over time and the change in activated electrodes for monitoring the bleeding event. The monitoring array may include electrodes that form four-point probes 780 with an integration axis 108 that extends across the blood vessel from a first electrode detector 115, to a second electrode detector 115′. Note that the electrode detectors 115, 115′ are configured between the respective electrode emitters 110, 110′ to form the four-point probe 780. Electrode emitter 110 may be an input electrode emitter and electrode emitter 110′ may be a return electrode emitter, to receive the emitter input from the input electrode emitter, and the two input electrode emitters may close an electrical loop for the electrical signal.

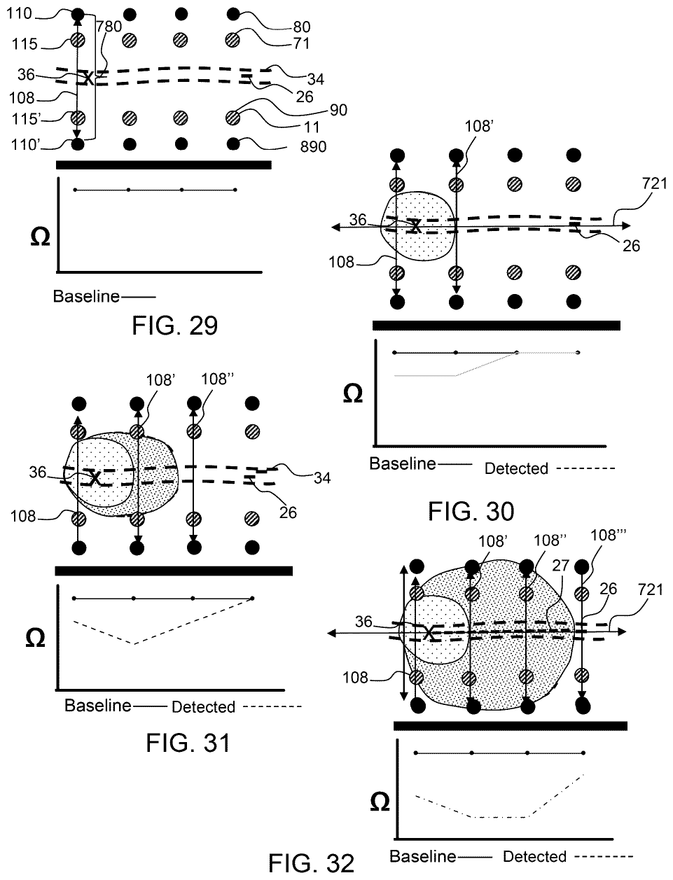

Referring now to FIGS. 29 to 32, monitoring arrays 71 with a plurality of emitters devices 80 and detectors devices 90 are configured on opposing sides of a blood vessel 34 and used for impedance measurements, and the respective graphs indicate changes in impedance as a bleeding event from the blood vessel progresses from FIG. 29 to FIG. 32. The emitters are in solid black and the detectors are shown with cross-hatching. The corresponding line graphs indicate a reduction in impedance over time for the interrogation axes of the pairs of emitters and detectors. FIG. 29 to FIG. 32 shows the progression over time from the baseline to the final detection determined by the four interrogation axes. The monitoring array may include electrodes that form four-point probes 780 with an integration axis 108 that extends across the blood vessel from a first electrode detector 115, to a second electrode detector 115′. Note that the electrode detectors 1150, 115′ are configured between the respective electrode emitters 110, 110′ to form the four-point probe 780. Note that the four-point probes extend across the blood vessel 34 but any of the transducers 890 may be activated to act as electrodes emitters or electrodes detectors enabling formation of four-point probes with interrogation axes that extend along the side of the blood vessel 34. The data shown represents the four four-point probes configured successively across the blood vessel. As shown in FIG. 32, four four-point probes 780 have been activated at separate times to produce four interrogation axes 108, 108′, 108″ and 108′″ that each extend across the length axis 721 and across the access track 27 from the skin incision 26 to the vessel puncture 36. These four interrogation axes extend across the length axis at offset distance from each other and as shown, may be parallel interrogation axes.

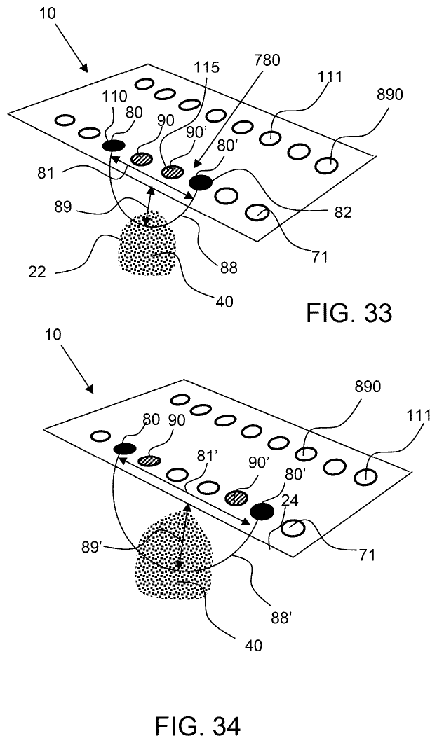

Referring now to FIGS. 33 and 34, a monitoring array 71 of transducers 890, which may be electrodes 111, are configured in rows. As shown in FIG. 33, a pair of emitter devices 80, 80′, electrode emitters 110, and pair of detector devices 90, 90′, electrode detectors 115, are activated to enable an emitter input depth 89 from the emitter input 88, an electrical signal. As shown in FIG. 33, this arrangement of electrode emitters and electrode detectors forms a four-point probe 780. In FIG. 33, the emitter devices 80, 80′ have an emitter offset distance 81, distance between the emitter devices, that is less than the emitter offset distance 81 shown in FIG. 34. In FIG. 34, a greater emitter input depth 89′, due to the greater emitter offset distance 81′, enables detection of the bleeding event or hematoma 40 at a greater depth from the skin surface 24 than the four-point probe 780 configuration in FIG. 33. Two different sizes of bleeding event are illustrated below the skin level in the cross-section view. The emitter inputs 88, 88′ are depicted as an arced line into the tissue 22 below the emitter array 82. The emitter input illustrates the field lines of current which may travel through the tissue. As electrodes with greater spacing are activated, the depth of penetration of the field lines increases, allowing for detection, characterization, and monitoring of bleeding events which are deeper below the surface of the skin. Thus, activation and deactivation of electrodes can optimize the depth of detection for bleeding events.

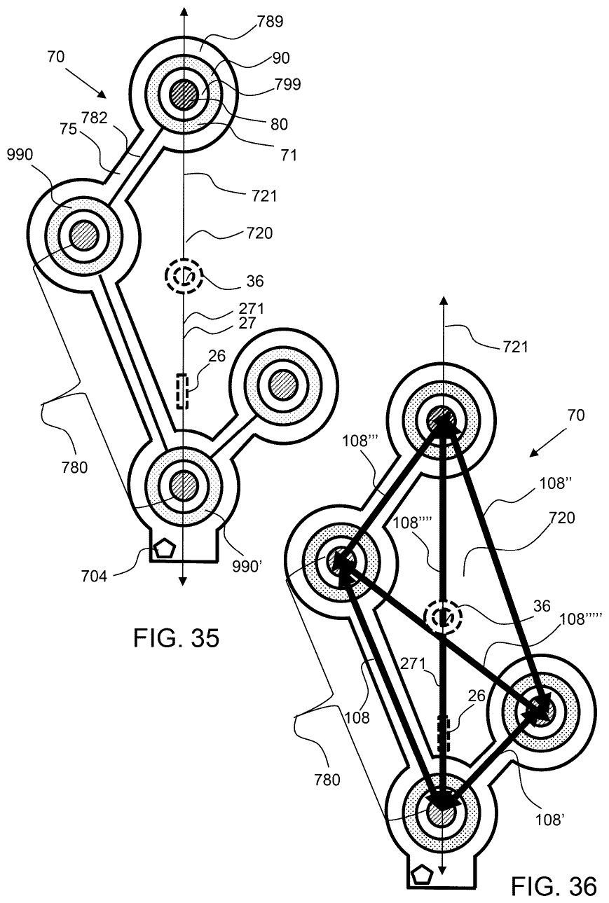

Referring now to FIGS. 35 to 38, a wearable monitor 70 of a wearable monitor system has a monitoring array 71 that has an monitor patch 75 with an open area 720 for accessing the skin incision 26 and/or access track 27 and the vessel puncture 36, and has electrical leads 782 that extend to monitoring hubs 789 configured around the vessel access site or open area. The open area 720 is formed by the arrangement of the monitoring hubs and connecting monitor patch 75 therebetween and is not a window open area enclosed by the monitor patch but a slot type open area. Each monitoring hub has an emitter device 80 and a detector device 90. As shown, the detector device 90 extends as a ring detector device around the central emitter device 80. Also, an insulating ring 799 may physically and/or electrically separate the emitter device 80 from the detector device 90. The monitoring hubs 789 are configured in an array with two configured on opposing sides of the length axis 721 of the wearable monitor 70 and two configured closer to the length axis and on opposing sides of the open area 720 along the length axis. Any two of the four monitoring hubs may be activated to form a four-point probe 780, wherein the two emitter devices 80 are outside of the two detector devices 90. In the four-point probe, a portion of the detector devices are configured between the two respective emitter devices. The emitter offset distance is the shortest distance between the two emitter devices, such as electrode emitters, and note that the detector offset distance, also the shortest distance between the two detector rings 990, 990′, such as electrode detectors, is less than the emitter offset distance. The ring configuration of the two detector rings ensures a portion of the emitter device is outside of the detector device along any interrogation axis activated. A controller is configured on the wearable monitor 70.

As shown in FIG. 36, the wearable monitor enables a plurality of interrogation axes 108, 108′, 108″, 108″′, 108″″, 108′″″, between different emitter devices or the six different four-point probe 780 configurations. As shown, four-point probe 780 has an integration axis 108 between the two central emitter devices. Note that the interrogation axes 108, 108′, 108″ and 108′″ form an interrogation perimeter that extends around a perimeter of the open area 720 and the vessel access area which may include the skin incision 26, access track 27 and the vessel puncture 36. As shown, the length axis 721 of the wearable monitor 70 is aligned along the access track 271. This perimeter arrangement of integration axes may more reliably enable early detection of a bleeding event. Furthermore, two additional interrogation axes 108″″ and 108″″′ extend through or across the open area 720, wherein interrogation axis 108″″′ extends across the length axis 721 and integration axis 108″″ extends along the length axis and access track 271 with the detector devices configured on opposing sides of the open area. There are four perimeter interrogation axes between the monitoring hubs that extend around a perimeter of the open area and the two traversing interrogation axes that extend across the open area 720 of the wearable monitor.

The configuration of the emitter devices and detector devices shown in FIGS. 35 and 36 may be effective for the NearIR and Ultrasound interrogation wearable monitors, wherein emitter devices may be configured in two of the monitoring hubs 789 and the detector devices may be configured in the remaining two monitoring hubs. It may be beneficial to have the emitter devices configured in the two monitoring hubs located on one end or region of the length axis and the detector devices configured on the opposing end of the length axis, wherein an emitter device is configured on one end of the length axis and a detector device is configured on the opposing end of the length axis, across the open area from the emitter device.

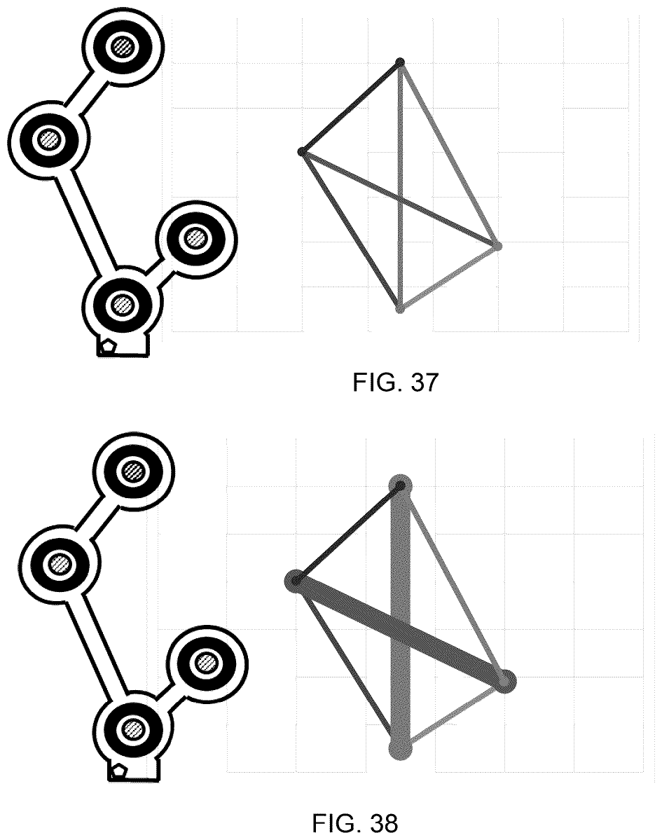

FIGS. 37 and 38 show graphical representations of the response signals wherein in FIG. 38 the two traversing interrogation axes across the length axis of the wearable monitor have significantly different values than these same interrogation axes shown in FIG. 37, indicating a bleeding event, wherein the larger width lines may indicate a drop in impedance. The four perimeter interrogation axes are unchanged from FIG. 37 to FIG. 38. FIG. 37 may represent a baseline impedance for each of the six four-point probes and FIG. 38 shows, by the weight of the integration lines, a drop in impedance in the open area that indicates a bleeding event.

It will be apparent to those skilled in the art that various modifications, combinations, and variations can be made in the present invention without departing from the scope of the invention. Specific embodiments, features and elements described herein may be modified, and/or combined in any suitable manner. Thus, it is intended that the present invention cover the modifications, combinations and variations of this invention provided they come within the scope of the appended claims and their equivalents.

What is claimed is:

1. A monitor system configured for the detection of bleeding events in tissue comprising:
a) a wearable monitor comprising:
i) a length axis;
ii) an interface side;
iii) a monitoring array comprising a plurality of transducers;
wherein the monitoring array includes a first four-point probe comprising:
two transducers of the plurality of transducers, each functioning as emitter devices that are electrodes to input an emitter input that is an electrical signal into said tissue,
two transducers of the plurality of transducers, each functioning as detector devices that are electrodes configured to detect a detection signal from the said emitter input of the first four-point probe;
wherein the two emitter devices are configured an emitter offset distance from each other along a first interrogation axis, and
b) an attachment component configured to detachably attach the monitor to a person with the interface side configured to face said tissue;
c) a controller comprising a computer;
wherein the controller controls activating and deactivating said emitter devices and activating and deactivating said detector devices;
wherein the controller receives said detection signal; and
d) an alert system comprising an alert device to produce said alert;
wherein the controller initiates said alert on said alert device when a change in the detection signal is above a threshold value; and
wherein the wearable monitor has an open area along the length axis to allow access to a skin incision in said tissue.

2. The monitor system of claim 1, wherein the detector devices have a detector offset distance that is less than the emitter offset distance.

3. The monitor system of claim 2, wherein the detector devices are configured between the emitter devices.

4. The monitor system of claim 1, wherein the monitoring array includes a second four-point probe comprising:
two transducers of the plurality of transducers, each functioning as emitter devices that are electrodes to input an emitter input that is an electrical signal into said tissue, and
two transducers of the plurality of transducers, each functioning as detector devices that are electrode configured to detect a detection signal from the said emitter input of the second four-point probe;
wherein the two emitter devices of the second four-point probe are configured a second emitter offset distance from each other along a second interrogation axis, and
wherein at least one of the emitter devices of the second four-point probe is different than the emitter devices of the first four-point probe.

5. The monitor system of claim 4, wherein one of the emitter devices of the second four-point probe utilizes one of the emitter devices of the first four-point probe, and wherein one of the detector devices of the second four-point probe utilizes one of the detector devices of the first four-point probe.

6. The monitor system of claim 4, wherein the controller switches off one of the emitter devices and one of the detector devices of the first four-point probe and activates one of the emitter devices and one of the detector devices of the second four-point probe to respectively deactivate the first four-point probe and activate the second four-point probe.

7. The monitor system of claim 4, wherein both of the emitter devices of the second four-point probe are different transducers than the emitter devices of the first four-point probe, and wherein both of the detector devices of the second four-point probe are different transducers than the detector devices of the first four-point probe.

8. The monitor system of claim 1, wherein the monitoring array comprises a plurality of four-point probes including the first four-point probe and wherein at least one of the plurality of four-point probes has an interrogation axis that extend across the length axis of the wearable monitor.

9. The monitor system of claim 8, wherein at least one of the plurality of four-point probes has an interrogation axis that extends on a first side of the length axis of the wearable monitor.

10. The monitor system of claim 9, wherein at least one of the plurality of four-point probes has an interrogation axis that extends on a second side, opposite the first side, of the length axis of the wearable monitor.

11. The monitor system of claim 10, wherein the plurality of four-point probes produce an interrogation perimeter with the interrogation axes of said plurality of four-point probes intersecting to form a monitored area within said interrogation perimeter, and wherein the interrogation perimeter extends around said open area.

12. The monitor system of claim 1, wherein the open area is a discrete open area wherein the wearable monitor extends around said discrete open area.

13. The monitor system of claim 1, wherein the controller activates and deactivates transducers of the plurality of transducers to function as emitter devices and detector devices to activate each of the four four-point probes to function independently.

14. The monitor system of claim 1, wherein the electrical signal has a frequency of 200 kHz or less.

15. The wearable monitor of claim 1, wherein the plurality of transducers includes at least eight transducers and includes a pair of transducers configured as a monitoring hub, wherein said monitoring hub includes a detector device configured as a detector ring around a central emitter device.

16. The wearable monitor of claim 15, wherein the monitoring hub further comprises an insulator ring between the detector ring and the central emitter device.

17. The wearable monitor of claim 15, wherein a pair of monitoring hubs produces said first four-point probe.

18. The wearable monitor of claim 1, wherein the wearable monitor further comprises a monitor patch and wherein the monitoring array is coupled to the monitor patch.

19. The wearable monitor of claim 1, further comprising a pump and a bladder that is configured to be inflated by said pump if a bleeding event is detected by a change in the detection signal of a diagnostic test.

20. The wearable monitor of claim 1, wherein the controller runs a calibration to determine a location of the access track, wherein one or more of the plurality of emitter devices are activated along with one or more of the plurality of detector devices to run a first calibration diagnostic and wherein one or more of the plurality of emitter devices are activated along with one or more of the plurality of detector devices to run a second calibration diagnostic, wherein the second calibration diagnostic activates different emitter devices than said first calibration diagnostic.
